SEQUENCE LISTING (20 sequences)

SEQ ID NO: 1 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 2 - DNA, 648 bp, Artificial Sequence
  humanized antibody light chain
gctatccagatgacccagtctccttcctccctgtctgcatctgtaggagacagagtcaccatcacttgccaggccagtcagagcattaacaatgagttatcctggtatcagcagaaaccagggaaagcccctaagctcctgatctatagggcatccactctggcatctggggtcccatcaaggttcagcggcagtggatctgggacagacttcactctcaccatcagcagcctgcagcctgatgattttgcaacttattactgccaacagggttatagtctgaggaacattgataatgctttcggcggagggaccaaggtggaaatcaaacgtgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 3 - protein, 216 aa, Artificial Sequence
  Synthetic Construct
AIQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIKRVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - DNA, 333 bp, Artificial Sequence
  Humanized Antibody Light Chain Variable Domain
gctatccagatgacccagtctccttcctccctgtctgcatctgtaggagacagagtcaccatcacttgccaggccagtcagagcattaacaatgagttatcctggtatcagcagaaaccagggaaagcccctaagctcctgatctatagggcatccactctggcatctggggtcccatcaaggttcagcggcagtggatctgggacagacttcactctcaccatcagcagcctgcagcctgatgattttgcaacttattactgccaacagggttatagtctgaggaacattgataatgctttcggcggagggaccaaggtggaaatcaaacgt

SEQ ID NO: 5 - protein, 111 aa, Artificial Sequence
  Synthetic Construct
AIQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIKR

SEQ ID NO: 6 - protein, 11 aa, Oryctolagus cuniculus
QASQSINNELS

SEQ ID NO: 7 - protein, 7 aa, Oryctolagus cuniculus
RASTLAS

SEQ ID NO: 8 - protein, 12 aa, Oryctolagus cuniculus
QQGYSLRNIDNA

SEQ ID NO: 9 - DNA, 315 bp, Homo sapiens
  CDS (1)..(315)
gtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 10 - protein, 105 aa, Homo sapiens [sequence omitted]

SEQ ID NO: 11 - DNA, 1350 bp, Artificial Sequence
  Humanized Antibody Heavy Chain
gaggtgcagctggtggagtctgggggaggcttggtccagcctggggggtccctgagactctcctgtgcagcctctggattctccctcagtaactactacgtgacctgggtccgtcaggctccagggaaggggctggagtgggtcggcatcatctatggtagtgatgaaaccgcctacgctacctccgctataggccgattcaccatctccagagacaattccaagaacaccctgtatcttcaaatgaacagcctgagagctgaggacactgctgtgtattactgtgctagagatgatagtagtgactgggatgcaaagttcaacttgtggggccaagggaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 12 - protein, 450 aa, Artificial Sequence
  Synthetic Construct
EVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 360 bp, Artificial Sequence
  Humanized Antibody Heavy Chain Variable Domain
gaggtgcagctggtggagtctgggggaggcttggtccagcctggggggtccctgagactctcctgtgcagcctctggattctccctcagtaactactacgtgacctgggtccgtcaggctccagggaaggggctggagtgggtcggcatcatctatggtagtgatgaaaccgcctacgctacctccgctataggccgattcaccatctccagagacaattccaagaacaccctgtatcttcaaatgaacagcctgagagctgaggacactgctgtgtattactgtgctagagatgatagtagtgactgggatgcaaagttcaacttgtggggccaagggaccctcgtcaccgtctcgagc

SEQ ID NO: 14 - protein, 120 aa, Artificial Sequence
  Synthetic Construct
EVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNLWGQGTLVTVSS

SEQ ID NO: 15 - protein, 5 aa, Oryctolagus cuniculus
NYYVT

SEQ ID NO: 16 - protein, 16 aa, Oryctolagus cuniculus
IIYGSDETAYATSAIG

SEQ ID NO: 17 - protein, 12 aa, Oryctolagus cuniculus
DDSSDWDAKFNL

SEQ ID NO: 18 - DNA, 990 bp, Homo sapiens
  CDS (1)..(990)
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 19 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - DNA, 483 bp, Pichia pastoris
  misc_feature (1)..(483): Glyceraldehyde-3-Phosphate dehydrogenase (GAP) promoter
agatcttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactat

(19) United States
(12) Patent Application Publication
(10) Pub. No.: US 2016/0024203 A1
(43) Pub. Date: Jan. 28, 2016

(54) METHODS OF PRODUCING ANTIBODIES IN YEAST

(71) Applicant: BRISTOL-MYERS SQUIBB COMPANY, Princeton, NJ (US)

(72) Inventors: Zizhuo Xing, Jamesville, NY (US); George S. Campbell, Fayetteville, NY (US); Bruce E. Eagan, Dewitt, NY (US); Yueming Qian, Manlius, NY (US); Xuankuo Xu, Manlius, NY (US); Li You, Jamesville, NY (US); Zhengjian Li, Sudbury, MA (US); Nan-Xin Qian, Manlius, NY (US)

(21) Appl. No.: 14/774,707

(22) PCT Filed: Mar. 14, 2014
(86) PCT No.: PCT/US2014/026949

§ 371 Date: Sep. 11, 2015

Related U.S. Application Data
(60) Provisional application No. 61/787,190, filed on Mar. 15, 2013.
(60) Provisional application No. 61/787,029, filed on Mar. 15, 2013.

(51) Int. Cl. C07K 16/24

(52) U.S. Cl. CPC C07K 16/248; C07K 2317/21; C07K 2317/24; C07K 2317/14

(57) ABSTRACT

The present invention describes a method for producing an antibody in Pichia pastoris, such as by fed-batch fermentation. The method includes the addition of about 2.0-5.0 g/L of hydroxyurea during the fermentation process to sustain a constant cell density and enhance the whole broth titer of the antibody. The method may also include a strategy of increasing the ethanol concentration to about 18-22 g/L and then maintaining the ethanol level at about 5-17 g/L to stabilize the cell mass and enhance the production rate of the antibody. The method may further include a respiratory quotient control for monitoring the ethanol profile and to improve the quality of the antibody by, for example, eliminating clipping of the heavy chain.

### CROSS-REFERENCE TO RELATED APPLICATIONS

The present application claims the benefit of U.S. Provisional Patent Application Ser. No. 61/787,190, filed Mar. 15, 2013, and U.S. Provisional Patent Application Ser. No. 61/787,029, filed Mar. 15, 2013, both of which are herein incorporated by reference in their entirety.

### BACKGROUND OF THE INVENTION

Antibodies have rapidly become a clinically important drug class: more than 25 antibodies are approved from human therapy and more than 240 antibodies are currently in clinical development worldwide for a wide range of disorders, including autoimmunity and inflammation, cancer, organ transplantation, cardiovascular disease, infectious diseases and ophthalmological diseases. Reichert, J. M., mAbs, 2:28-45 (2010); Chan et al., Nature Reviews Immunology, 10(5):301-316 (May 2010). The clinical success of antibodies has led to a major commercial impact, with rapidly growing annual sales that exceeded US $27 billion in 2007, including 8 of the 20 top-selling biotechnology drugs. Scolnik, P. A., mAbs, 1:179-184 (2009); and Chan et al., Nature Reviews Immunology, 10(5):301-316 (May 2010).

For some time, mammalian cells have served as the major hosts for antibody production, irrespective of their high cost and the long periods required for cultivation. However, as demand for antibody therapeutics increases, the economics associated with production an antibodies becomes an important issue. Consequently, continuing interest exists in devising superior and more affordable processes that employ simple cost-effective hosts, such as yeast, e.g., Saccharomyces cerevisiae or Pichia pastoris, instead of mammalian cells. Jeong et al., Biotechnology J., 6(1):16-27 (January 2011).

Hydroxyurea was used as stress-inducing compounds in yeast fermentation (Schmitt et al., Appl. Env. Microbiol., 72:1515-1522 (2006)). Specifically, Doran et al. (Doran, P. M. et al., Biotechnol. Bioeng., 28:1814-1831 (1986)) reported morphological and physiological response of suspended S. cerevisiae cells on the addition of 5.7 g/L hydroxyurea. The cell population was arrested by hydroxyurea, which resulted in reduction of cell mass by 50% and total polysaccharide content by 65%. There was an accumulation of suspended cells with large buds. Under the stress introduced by hydroxyurea, cells had increased specific glucose consumption rate and ethanol production rate. However, synthesis of protein and RNA was not adversely affected.

The present invention relates to an improved process for producing a higher quantity of antibodies or antigen-binding fragments using yeast. The process includes the addition of 2.0-5.0 g/L of hydroxyurea during the fermentation process to sustain a constant cell density and enhance the whole broth titer of the antibody. The present invention, as set forth herein, meets these and other needs.

### BRIEF DESCRIPTION OF THE DRAWINGS

FIG. 1 illustrates the fermentation process scheme for production of an antibody or an antigen-binding fragment thereof.

FIG. 2 shows the residual ethanol concentrations of the fermentation experiments of FIG. 1.

FIG. 3 shows the wet cell weight of the fermentation experiments of FIG. 1.

FIG. 4 shows the supernatant titer of the fermentation experiments of FIG. 1.

FIG. 5 shows the whole broth titer of the fermentation experiments of FIG. 1.

FIG. 6 shows the specific antibody production rates (based on wet cell weight) of the fermentation experiments of FIG. 1.

FIG. 7 shows the ethanol of Run 01MAY11 in Example 2.

FIG. 8 shows the wet cell weight (WCW) of Run 01MAY11 in Example 2.

FIG. 9 shows the supernatant titer of Run 01MAY11 in Example 2.

FIG. 10 shows the whole broth (WB) titer of Run 01MAY11 in Example 2.

FIG. 11 shows the antibody protein product rate (based on wet cell weight) of Run 01MAY11 in Example 2.

FIG. 12 shows the RQ profiles of fermentation runs of Example 3. The horizontal line indicates the RQ value of 1.1. The vertical line indicates the latest time of the cultures entering the ethanol stabilization period (Lot 16MAY11T5). The period with a cross inside of a circle indicates values greater than 1.1.

FIG. 13 shows the ethanol profiles of fermentation runs of Example 3. The vertical line indicates the latest time of the cultures entering the ethanol stabilization period (Lot 16MAY11T5).

FIG. 14 shows the wet cell weight (WCW) profiles of fermentation runs of Example 3. The vertical line indicates the latest time of the cultures entering the ethanol stabilization period (Lot 16MAY11T5).

FIG. 15 shows non-reduced and reduced SDS-PAGE gels in Example 3 that demonstrated detectable level (Lot 01MAY11T5) or below detectable level of 37 kD and 19 kD bands by compared to the band of 0.05 μg BSA.

FIG. 16 shows RQ profiles of fermentation runs of Run 19JUL11 in Example 4. The horizontal line indicates the RQ value of 1.1. The vertical line demonstrates the latest time of the cultures entering the ethanol stabilization period (Lot 19JUN11T2 and T9). The period with a cross inside of a circle indicates values greater than 1.1.

FIG. 17 shows ethanol profiles of Run 19JUL11 in Example 4. The vertical line demonstrates the latest time of the cultures entering the ethanol stabilization period (Lot 19JUN11T2 and T9).

FIG. 18 shows wet cell weight (WCW) profiles of Run 19JUL11 in Example 4. The vertical line demonstrates the latest time of the cultures entering the ethanol stabilization period (Lot 19JUN11T2 and T9).

FIG. 19 shows non-reduced and reduced SDS-PAGE gels that demonstrate purified antibody with or without 37/19 kD bands in Example 4. The detectable levels of 37 kD and 19 kD bands were determined by comparing the bands to the band of 0.05 μg BSA.

FIG. 20 shows reducing SDS-PAGE gels that demonstrates purified antibody of Lot 01MAY11T5 with 37/19 kD bands for N-terminal sequencing in Example 5.

FIG. 21 shows non-reduced and reduced SDS-PAGE gels of the antibody for Example 6.

FIG. 22 shows the engineering parameters of the three consistent lots of the fermentation experiments of FIG. 1.

FIG. 23 shows the air flow profiles of the three consistent lots of the fermentation experiments of FIG. 1.

FIG. 24 shows feeding profiles of the three consistent lots of the fermentation experiments of FIG. 1.

FIG. 25 shows glucose profiles of the three consistent lots of the fermentation experiments of FIG. 1.

FIG. 26 shows RQ profiles of the three consistent lots of the fermentation experiments of FIG. 1.

FIG. 27 shows ethanol profiles of the three consistent lots of the fermentation experiments of FIG. 1.

FIG. 28 shows wet cell weight (WCW) profiles of the three consistent lots of the fermentation experiments of FIG. 1.

FIG. 29 shows supernatant titer profiles of the three consistent lots of the fermentation experiments of FIG. 1.

FIG. 30 shows whole broth (WB) titer profiles of the three consistent lots of the fermentation experiments of FIG. 1.

### DETAILED DESCRIPTION OF THE INVENTION

### Definitions

In the description that follows, a number of terms are used extensively. The following definitions are provided to facilitate understanding of the invention.

Unless otherwise specified, “a”, “an”, “the”, and “at least one” are used interchangeably and mean one or more than one.

“Antibodies” (Abs) and “immunoglobulins” (Igs) are glycoproteins having the same structural characteristics. While antibodies or antigen-binding fragments thereof exhibit binding specificity to a specific antigen, immunoglobulins include both antibodies and other antibody-like molecules that lack antigen specificity. Polypeptides of the latter kind are, for example, produced at low levels by the lymph system and at increased levels by myelomas. Thus, as used herein, the term “antibody” or “antibody peptide(s)” refers to an intact antibody, or an antigen-binding fragment thereof that competes with the intact antibody for specific binding and includes chimeric, humanized, fully human, and bispecific antibodies. In certain embodiments, binding fragments are produced, for example, by recombinant DNA techniques. In additional embodiments, binding fragments are produced by enzymatic or chemical cleavage of intact antibodies. Antigen-binding fragments include, but are not limited to, Fab, Fab′, F(ab)₂, F(ab′)₂, Fv, domain antibodies, and single-chain antibodies.

An “isolated antibody” as used herein refers to an antibody that has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In other embodiments, the antibody will be purified (1) to greater than 95% by weight of antibody as determined by the Lowry method, and may be more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

A “bispecific” or “bifunctional” antibody is a hybrid antibody having two different heavy/light chain pairs and two different binding sites. Bispecific antibodies may be produced by a variety of methods including, but not limited to, fusion of hybridomas or linking of Fab′ fragments. See, e.g., Songsivilai et al., Clin. Exp. Immunol., 79:315-321 (1990); Kostelny et al., J. Immunol., 148:1547-1553 (1992).

As used herein, the term “epitope” refers to the portion of an antigen to which an antibody specifically binds. Thus, the term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. An epitope having immunogenic activity is a portion of target polypeptide or antigen, such as a cytokine, e.g., IL-6, a cytokine receptor or cell surface receptor or cell surface protein that elicits an antibody response in an animal. An epitope having antigenic activity is a portion of the target polypeptide or antigen to which an antibody immunospecifically binds as determined by any method well known in the art, for example, by immunoassays, protease digest, crystallography or H/D-Exchange. Antigenic epitopes need not necessarily be immunogenic. Such epitopes can be linear in nature or can be a discontinuous epitope. Thus, as used herein, the term “conformational epitope” refers to a discontinuous epitope formed by a spatial relationship between amino acids of an antigen other than an unbroken series of amino acids.

As used herein, the term “immunoglobulin” refers to a protein consisting of one or more polypeptides substantially encoded by immunoglobulin genes. One form of immunoglobulin constitutes the basic structural unit of an antibody. This form is a tetramer and consists of two identical pairs of immunoglobulin chains, each pair having one light and one heavy chain. In each pair, the light and heavy chain variable regions are together responsible for binding to an antigen, and the constant regions are responsible for the antibody effector functions.

Full-length immunoglobulin “light chains” (about 25 kD or about 214 amino acids) are encoded by a variable region gene at the NH₂-terminus (about 110 amino acids) and a kappa or lambda constant region gene at the COOH-terminus. Full-length immunoglobulin “heavy chains” (about 50 kD or about 446 amino acids), are similarly encoded by a variable region gene (about 116 amino acids) and one of the other aforementioned constant region genes (about 330 amino acids). Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” region of about 12 or more amino acids, with the heavy chain also including a “D” region of about 10 more amino acids. (See generally, Fundamental Immunology, Ch. 7 (Paul, W., ed., 2nd Edition, Raven Press, N.Y. (1989)), (incorporated by reference in its entirety for all purposes).

The term “cytokine” is a generic term for proteins or peptides released by one cell population which act on another cell as intercellular mediators. As used broadly herein, examples of cytokines include lymphokines, monokines, growth factors and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-.alpha. and -.beta.; mullerian-inhibiting substance; mouse gonadotropin-associated peptide; inhibin; activin; vascular endothelial growth factor; integrin; thrombopoietin (TPO); nerve growth factors such as NGF-.beta.; platelet-growth factor; transforming growth factors (TGFs) such as TGF-.alpha. and TGF-.beta.; insulin-like growth factor-I and -II; erythropoietin (EPO); osteoinductive factors; interferons such as interferon-alpha., -beta., and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1. alpha., IL-2, IL-3, IL-4, IL-5, IL-6, IL-7, IL-8, IL-9, IL-10, IL-11, IL-12; IL-13, IL-14, IL-15, IL-16, IL-17, IL-18, IL-21, IL-22, IL-23, IL-27, IL-28, IL-29, IL-31, IL-32, IL-33, IL-34, IL-35, IL-36, ILLIF, G-CSF, GM-CSF, M-CSF, EPO, kit-ligand or FLT-3, angiostatin, thrombospondin, endostatin, tumor necrosis factor and LT.

An immunoglobulin light or heavy chain variable region consists of a “framework” region interrupted by three hypervariable regions. Thus, the term “hypervariable region” refers to the amino acid residues of an antibody which are responsible for antigen binding. The hypervariable region comprises amino acid residues from a “Complementarity Determining Region” or “CDR” (Kabat et al., Sequences of Proteins of Immunological Interest, 5th Edition, Public Health Service, National Institutes of Health, Bethesda, Md. (1991)) and/or those residues from a “hypervariable loop” (Chothia et al., J. Mol. Biol., 196:901-917 (1987)) (both of which are incorporated herein by reference). “Framework Region” or “FR” residues are those variable domain residues other than the hypervariable region residues as herein defined. The sequences of the framework regions of different light or heavy chains are relatively conserved within a species. Thus, a “human framework region” is a framework region that is substantially identical (about 85% or more, usually 90-95% or more) to the framework region of a naturally occurring human immunoglobulin. The framework region of an antibody, that is the combined framework regions of the constituent light and heavy chains, serves to position and align the CDR's. The CDR's are primarily responsible for binding to an epitope of an antigen. Accordingly, the term “humanized” immunoglobulin refers to an immunoglobulin comprising a human framework region and one or more CDR's from a non-human (usually a mouse or rat) immunoglobulin. The non-human immunoglobulin providing the CDR's is called the “donor” and the human immunoglobulin providing the framework is called the “acceptor”. Constant regions need not be present, but if they are, they must be substantially identical to human immunoglobulin constant regions, i.e., at least about 85-90%, preferably about 95% or more identical. Hence, all parts of a humanized immunoglobulin, except possibly the CDR's, are substantially identical to corresponding parts of natural human immunoglobulin sequences. Further, residues in the human framework region may be back mutated to the parental sequence to retain optimal antigen-binding affinity and specificity. In this way, certain framework residues from the non-human parent antibody are retained in the humanized antibody in order to retain the binding properties of the parent antibody while minimizing its immunogenicity. The term “human framework region” as used herein includes regions with such back mutations. A “humanized antibody” is an antibody comprising a humanized light chain and a humanized heavy chain immunoglobulin. For example, a humanized antibody would not encompass a typical chimeric antibody as defined above, e.g., because the entire variable region of a chimeric antibody is non-human.

The term “humanized” immunoglobulin refers to an immunoglobulin comprising a human framework region and one or more CDR's from a non-human (usually a mouse or rat) immunoglobulin. The non-human immunoglobulin providing the CDR's is called the “donor” and the human immunoglobulin providing the framework is called the “acceptor”. Constant regions need not be present, but if they are, they must be substantially identical to human immunoglobulin constant regions, i.e., at least about 85-90%, preferably about 95% or more identical. Hence, all parts of a humanized immunoglobulin, except possibly the CDR's and possibly a few back-mutated amino acid residues in the framework region (e.g., 1-10 residues), are substantially identical to corresponding parts of natural human immunoglobulin sequences. A “humanized antibody” is an antibody comprising a humanized light chain and a humanized heavy chain immunoglobulin. For example, a humanized antibody would not encompass a typical chimeric antibody as defined above, e.g., because the entire variable region of a chimeric antibody is non-human.

As used herein, the term “human antibody” includes an antibody that has an amino acid sequence of a human immunoglobulin and includes antibodies isolated from human immunoglobulin libraries or from animals transgenic for one or more human immunoglobulin and that do not express endogenous immunoglobulins, as described, for example, by Kucherlapati et al. in U.S. Pat. No. 5,939,598.

A “Fab fragment” is comprised of one light chain and the C_{H1} and variable regions of one heavy chain. The heavy chain of a Fab molecule cannot form a disulfide bond with another heavy chain molecule.

A “Fab′ fragment” contains one light chain and one heavy chain that contains more of the constant region, between the C_{H1} and C_{H2} domains, such that an interchain disulfide bond can be formed between two heavy chains to form a F(ab′)₂ molecule.

A “F(ab′)₂ fragment” contains two light chains and two heavy chains containing a portion of the constant region between the C_{H1} and C_{H2} domains, such that an interchain disulfide bond is formed between two heavy chains.

A “Fv fragment” contains the variable regions from both heavy and light chains but lacks the constant regions.

A “single domain antibody” is an antibody fragment consisting of a single domain Fv unit, e.g., V_{H} or V_{L}. Like a whole antibody, it is able to bind selectively to a specific antigen. With a molecular weight of only 12-15 kD, single-domain antibodies are much smaller than common antibodies (150-160 kD) which are composed of two heavy protein chains and two light chains, and even smaller than Fab fragments (˜50 kD, one light chain and half a heavy chain) and single-chain variable fragments (˜25 kD, two variable domains, one from a light and one from a heavy chain). The first single-domain antibodies were engineered from heavy-chain antibodies found in camelids. Although most research into single-domain antibodies is currently based on heavy chain variable domains, light chain variable domains and nanobodies derived from light chains have also been shown to bind specifically to target epitopes.

The term “monoclonal antibody” as used herein refers to an antibody or antigen-binding fragment thereof that is derived from a single clone, including any eukaryotic, prokaryotic, or phage clone, and not the method by which it is produced.

As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids”, which comprise naturally-occurring or modified nucleic acid bases attached to a polyamide backbone. Nucleic acids can be either single stranded or double stranded.

The term “complement of a nucleic acid molecule” refers to a nucleic acid molecule having a complementary nucleotide sequence and reverse orientation as compared to a reference nucleotide sequence.

“Respiratory Quotient” or “RQ” refers to the ratio of carbon dioxide produced to oxygen consumed, i.e., CO₂ produced/O₂ consumed.

“Batch fermentation conditions” refer to a closed loop culture system in which the microorganism(s) (inoculums) and nutrients are added at the beginning of fermentation, nothing is added or removed during the fermentation (except, for example, venting of waste gas, reagents for pH adjustment, and samples for assay), and the culture is harvested at the end of fermentation when the nutrients are depleted. The volume of the fermentation broth does not increase during batch fermentation.

“Fed-batch fermentation conditions” refer to an open loop culture system which includes a batch phase and a feeding phase. Fed-batch fermentation is started from a batch culture phase. Fresh medium is fed to the culture system when nutrients are depleted. The culture is not removed during fermentation (except, for example, removing a sample to test in an assay). It results in continuous increase in volume of the fermentation broth.

The term “degenerate nucleotide sequence” denotes a sequence of nucleotides that includes one or more degenerate codons as compared to a reference nucleic acid molecule that encodes a polypeptide. Degenerate codons contain different triplets of nucleotides, but encode the same amino acid residue (e.g., GAU and GAC triplets each encode Asp). As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids”, which comprise naturally-occurring or modified nucleic acid bases attached to a polyamide backbone. Nucleic acids can be either single stranded or double stranded.

“Complementary DNA (cDNA)” is a single-stranded DNA molecule that is formed from an mRNA template by the enzyme reverse transcriptase. Typically, a primer complementary to portions of mRNA is employed for the initiation of reverse transcription. Those skilled in the art also use the term “cDNA” to refer to a double-stranded DNA molecule consisting of such a single-stranded DNA molecule and its complementary DNA strand. The term “cDNA” also refers to a clone of a cDNA molecule synthesized from an RNA template.

A “promoter” is a nucleotide sequence that directs the transcription of a structural gene. Typically, a promoter is located in the 5′ non-coding region of a gene, proximal to the transcriptional start site of a structural gene, such as the glyceraldehydes-3-phosphate (GAP) transcription promoter. Sequence elements within promoters that function in the initiation of transcription are often characterized by consensus nucleotide sequences. These promoter elements include RNA polymerase binding sites, TATA sequences, CAAT sequences, differentiation-specific elements (DSEs; McGehee et al., Mol. Endocrinol., 7:551 (1993)), cyclic AMP response elements (CREs), serum response elements (SREs; Treisman, Seminars in Cancer Biol., 1:47 (1990)), glucocorticoid response elements (GREs), and binding sites for other transcription factors, such as CRE/ATF (O'Reilly et al., J. Biol. Chem., 267:19938 (1992)), AP2 (Ye et al., J. Biol. Chem., 269:25728 (1994)), SP1, cAMP response element binding protein (CREB; Loeken, Gene Expr., 3:253 (1993)) and octamer factors (see, in general, Watson et al., eds., Molecular Biology of the Gene, 4th Edition (The Benjamin/Cummings Publishing Company, Inc. (1987)), and Lemaigre et al., Biochem. J., 303:1 (1994)). If a promoter is an inducible promoter, then the rate of transcription increases in response to an inducing agent. In contrast, the rate of transcription is not regulated by an inducing agent if the promoter is a constitutive promoter. Repressible promoters are also known.

A “regulatory element” is a nucleotide sequence that modulates the activity of a core promoter. For example, a regulatory element may contain a nucleotide sequence that binds with cellular factors enabling transcription exclusively or preferentially in particular cells, tissues, or organelles. These types of regulatory elements are normally associated with genes that are expressed in a “cell-specific”, “tissue-specific”, or “organelle-specific” manner.

A “DNA segment” is a portion of a larger DNA molecule having specified attributes. For example, a DNA segment encoding a specified polypeptide is a portion of a longer DNA molecule, such as a plasmid or plasmid fragment, that when read from the 5′ to the 3′ direction, encodes the sequence of amino acids of the specified polypeptide.

“Heterologous DNA” refers to a DNA molecule, or a population of DNA molecules, that does not exist naturally within a given host cell. DNA molecules heterologous to a particular host cell may contain DNA derived from the host cell species (i.e., endogenous DNA) so long as that host DNA is combined with non-host DNA (i.e., exogenous DNA). For example, a DNA molecule containing a non-host DNA segment encoding a polypeptide operably linked to a host DNA segment comprising a transcription promoter is considered to be a heterologous DNA molecule. Conversely, a heterologous DNA molecule can comprise an endogenous gene operably linked with an exogenous promoter. As another illustration, a DNA molecule comprising a gene derived from a wild-type cell is considered to be heterologous DNA if that DNA molecule is introduced into a mutant cell that lacks the wild-type gene.

An “expression vector” is a nucleic acid molecule encoding an antibody or antigen-binding fragment thereof that is expressed in a host cell. Typically, an expression vector comprises a transcription promoter, a polynucleotide or DNA segment encoding an antibody or antigen-binding fragment thereof, and a transcription terminator. Gene expression is usually placed under the control of a promoter, and such a gene is said to be “operably linked to” the promoter. Similarly, a regulatory element and a core promoter are operably linked if the regulatory element modulates the activity of the core promoter.

A “recombinant host” is a cell that contains a heterologous nucleic acid molecule, such as a cloning vector or expression vector. In the present context, an example of a recombinant host is a cell that produces an antibody or antigen-binding fragment thereof from an expression vector.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

### Hydroxyurea

Exemplary hydroxyurea includes, but is not limited to, for example, 1-Hydroxyurea, 1-hydroxyurea, 4-03-00-00170 (Beilstein Handbook Reference), AI3-51139, BRN 1741548, Biosupressin, CCRIS 958, Carbamohydroxamic acid, Carbamohydroximic acid, Carbamohydroxyamic acid, Carbamoyl oxime, Carbamyl hydroxamate, DRG-0253, Droxia, HSDB 6887, HU, Hidrix, Hidroksikarbamid, Hidroksikarbamidas, Hidroxicarbamida, Hidroxikarbamid, Hydoxyurea, Hydrea, Hydreia, Hydroksikarbamidi, Hydroksiüre, Hydroxicarbamidum, Hydroxikarbamid, Hydroxy urea (d4), Hydroxycarbamide, Hydroxycarbamide-Addmedica, Hydroxycarbamidum, Hydroxycarbamine, Hydroxyharnstoff, “Hydroxylamine, N-(aminocarbonyl)-”, “Hydroxylamine, N-carbamoyl-”, Hydroxylurea, Hydroxymocovina, Hydroxyurea, Hydroxyurea (D4), Hydroxyurea (USAN), Hydroxyurea-Addmedica, Hydura, Hydurea, Idrossicarbamide, Litaler, Litalir, N-(Aminocarbonyl)hydroxylamine, N-(aminocarbonyl)hydroxylamine, N-Carbamoylhydroxylamine, N-Hydroxyurea, NCI-C04831, NSC 32065, NSC-32065, Onco-Carbide, Onco-carbide, OncoCarbide, Oxyurea, SK 22591, SQ 1089, SQ-1089, Siklos, “Urea, hydroxy-(8CI 9CI)”, WLN: ZVMQ, WR 83799, WR-83799, hydroxy urea (d4), sk 22591, sq 1089, wr 83799.

### Yeast Strain for the Production of Heterologous Antibodies

The antibody or antigen-binding fragment is a genetically engineered antibody that is directed against a polypeptide, such as a cytokine, e.g., Interleukins such as IL-6, or a receptor, e.g., cell surface receptors, cytokine receptor, interleukin receptors or chemokine receptors. The antibody, for instance, is composed of two identical heavy chains and two identical light chains. Briefly, the DNA sequence encoding light chain was inserted into the glyceraldehyde-3-phosphate dehydrogenase (GAP) promoter expression cassette of a haploid, while the DNA sequence encoding the heavy chain was inserted into the GAP promoter expression cassette of another haploid of P. pastoris. The two types of haploids were then mated to produce single colonies of diploid. A candidate of the production strain was propagated from each single colony. After screening, the production strain was selected for its high productivity with desired product quality. The yeast cells may, optionally, be of Pichia pastoris, Pichia methanolica, Pichia angusta, Pichia thermomethanolica or Saccharomyces cerevisiae. Optionally, the DNA segment encoding the heavy chain polypeptide and the light chain polypeptide are both operably linked to the same GAP promoter. Optionally, the DNA segment encoding the heavy chain polypeptide is operably linked to a first GAP promoter and the DNA segment encoding the light chain polypeptide is operably linked to a second GAP promoter. The GAP promoter may be derived from Pichia pastoris. The GAP promoter may have the nucleotide sequence of SEQ ID NO:20. The antibody or antigen-binding fragment thereof may specifically bind a cytokine (e.g., IL-6), receptor (e.g., chemokine receptor, cell surface receptor, interleukin receptor or a cytokine receptor) or a cell surface protein. Optionally, the antibody or antigen-binding fragment may be monoclonal or polyclonal. Optionally, the antibody or antigen-binding fragment may be multivalent, such as, for instance, a bispecific antibody. Optionally, the antibody may be a chimeric antibody, a human antibody or humanized antibody. Optionally, the antigen-binding fragment is Fab, Fab′, F(ab)₂, F(ab)₂, Fv or a single-chain Fv. Optionally, the antibody is an anti-human IL-6 monoclonal antibody, which may be a humanized anti-human IL-6 monoclonal antibody. The antibody may comprise a light chain polypeptide which comprises a light chain variable domain comprising the following CDRs: CDR1 having the amino acid sequence of SEQ ID NO:6; CDR2 having the amino acid sequence of SEQ ID NO:7; and CDR3 having the amino acid sequence of SEQ ID NO:8. The antibody may comprise a heavy chain polypeptide which comprises a heavy chain variable domain comprising the following CDRs: CDR1 having the amino acid sequence of SEQ ID NO:15; CDR2 having the amino acid sequence of SEQ ID NO:16; and CDR3 having the amino acid sequence of SEQ ID NO:17. Optionally, the antibody comprises a light chain polypeptide comprising a light chain variable domain comprising the following CDRs: CDR1 having the amino acid sequence of SEQ ID NO:6; CDR2 having the amino acid sequence of SEQ ID NO:7; and CDR3 having the amino acid sequence of SEQ ID NO:8; and a heavy chain polypeptide comprising a heavy chain variable domain comprising the following CDRs: CDR1 having the amino acid sequence of SEQ ID NO:15; CDR2 having the amino acid sequence of SEQ ID NO:16; and CDR3 having the amino acid sequence of SEQ ID NO:17. Optionally, the antibody may comprise a light chain variable domain comprising the amino acid sequence of SEQ ID NO:5. Optionally, the antibody may comprise a heavy chain variable domain comprising the amino acid sequence of SEQ ID NO:14. Optionally, the antibody comprises a light chain variable domain comprising the amino acid sequence of SEQ ID NO:5, and a heavy chain variable domain comprising the amino acid sequence of SEQ ID NO:14. The antibody may comprise or the antigen-binding fragment may further comprise a human heavy chain immunoglobulin constant domain of IgG, IgM, IgE or IgA, wherein the human IgG heavy chain immunoglobulin constant domain can be IgG1, IgG2, IgG3 or IgG4.

### Fermentation Process for the Production of Heterologous Antibodies

The antibody is produced in fermentation using the production strain. The fermentation process is initiated, for example, from thawing a frozen vial of a cell bank, which includes two steps of shake flask seed cultures to propagate cells and the main culture step in a bioreactor for the antibody production. Supernatant of the main culture is then harvested for downstream purification. The seed cultures are batch mode fermentation, while the main culture uses a novel fermentation process as described herein. One aspect of the novel fermentation process as described herein includes the addition of hydroxyurea to enhance antibody productivity by increasing integrated wet cell weight, and/or a unique ethanol control strategy to balance cell growth and the specific antibody production rate, and/or a RQ control strategy to maintain optimum ethanol profile and improve product quality.

The present invention provides a method for producing an antibody or antigen-binding fragment thereof in yeast comprising: a) providing a population of cultured yeast cells, wherein each cell comprises a DNA segment encoding a heavy chain polypeptide and a light chain polypeptide of the antibody operably linked to a glyceraldehyde-3-phosphate (GAP) transcription promoter and a transcription terminator; b) culturing the cells of step (a) under batch fermentation conditions; c) culturing the cells of step (b) under fed-batch fermentation conditions comprising administering 2.0-5.0 g/L of hydroxyurea to the cell culture at about 12-30 hours of the fermentation process; d) harvesting the cells of step (c) at 100-140 hours of the fermentation process; and e) recovering the antibody produced by the harvested cells of step (d). The yeast cells may, optionally, be of Pichia pastoris, Pichia methanolica, Pichia angusta, Pichia thermomethanolica or Saccharomyces cerevisiae. Optionally, the DNA segment encoding the heavy chain polypeptide and the light chain polypeptide are both operably linked to the same GAP promoter. Optionally, the DNA segment encoding the heavy chain polypeptide is operably linked to a first GAP promoter and the DNA segment encoding the light chain polypeptide is operably linked to a second GAP promoter. The GAP promoter may be derived from Pichia pastoris. The GAP promoter may have the nucleotide sequence of SEQ ID NO:20. The antibody or antigen-binding fragment thereof may specifically bind a cytokine (e.g., IL-6), receptor (e.g., chemokine receptor, cell surface receptor, interleukin receptor or a cytokine receptor) or a cell surface protein. Optionally, the antibody or antigen-binding fragment may be monoclonal or polyclonal. Optionally, the antibody or antigen-binding fragment may be multivalent, such as, for instance, a bispecific antibody. Optionally, the antibody may be a chimeric antibody, a human antibody or humanized antibody. Optionally, the antigen-binding fragment is Fab, Fab′, F(ab)₂, F(ab)₂, Fv or a single-chain Fv. Optionally, the antibody is an anti-human IL-6 monoclonal antibody, which may be a humanized anti-human IL-6 monoclonal antibody. The antibody may comprise a light chain polypeptide which comprises a light chain variable domain comprising the following CDRs: CDR1 having the amino acid sequence of SEQ ID NO:6; CDR2 having the amino acid sequence of SEQ ID NO:7; and CDR3 having the amino acid sequence of SEQ ID NO:8. The antibody may comprise a heavy chain polypeptide which comprises a heavy chain variable domain comprising the following CDRs: CDR1 having the amino acid sequence of SEQ ID NO:15; CDR2 having the amino acid sequence of SEQ ID NO:16; and CDR3 having the amino acid sequence of SEQ ID NO:17. Optionally, the antibody comprises a light chain polypeptide comprising a light chain variable domain comprising the following CDRs: CDR1 having the amino acid sequence of SEQ ID NO:6; CDR2 having the amino acid sequence of SEQ ID NO:7; and CDR3 having the amino acid sequence of SEQ ID NO:8; and a heavy chain polypeptide comprising a heavy chain variable domain comprising the following CDRs: CDR1 having the amino acid sequence of SEQ ID NO:15; CDR2 having the amino acid sequence of SEQ ID NO:16; and CDR3 having the amino acid sequence of SEQ ID NO:17. Optionally, the antibody may comprise a light chain variable domain comprising the amino acid sequence of SEQ ID NO:5. Optionally, the antibody may comprise a heavy chain variable domain comprising the amino acid sequence of SEQ ID NO:14. Optionally, the antibody comprises a light chain variable domain comprising the amino acid sequence of SEQ ID NO:5, and a heavy chain variable domain comprising the amino acid sequence of SEQ ID NO:14. The antibody may comprise or the antigen-binding fragment may further comprise a human heavy chain immunoglobulin constant domain of IgG, IgM, IgE or IgA, wherein the human IgG heavy chain immunoglobulin constant domain can be IgG1, IgG2, IgG3 or IgG4. Optionally, part (c) of the method comprises adding about 2.0-4.5 g/L, about 2.0-4.0 g/L, about 3.0-4.0 g/L, about 2.5-5.0 g/L, about 2.1-2.9 g/L, about 2.2-2.8 g/L, about 2.6-2.8 g/L, about 2.5-2.8 g/L, about 2.6-2.9 g/L, about 2.3-2.7 g/L, about 2.4-2.6 g/L or about 2.5 of hydroxyurea at about 12-30 hours, 14-19 hours, 16-21 hours or about 16-22 hours of the fermentation process. Optionally, the method may further comprise a step of adjusting a first respiratory quotient (RQ1) to about 1.1-1.6, to about 1.1-1.5, to about 1.2-1.6, to about 1.2-1.5, to about 1.3-1.4, or about 1.25-1.45 at about 20-40/48 hours of the fermentation process. Optionally, the RQ1 is adjusted to about 1.1-1.6 to increase the concentration of ethanol to about 15-23 g/L, about 17-23 g/L, about 17-22 g/L, about 18-22 g/L or about 19-21 g/L of the cell culture at 40/48 hour of the fermentation process. Optionally, the method may further comprise a step of adjusting a second respiratory quotient (RQ2) to about 0.8-1.1, to about 0.8-1.15, to about 0.85-1.1, to about 0.85-1.15, to about 0.9-1.1, to about 0.9-1.15, to about 0.95-1.1, or to about 0.95-1.15 at 40/48-100/140 hours of the fermentation process. Optionally, the RQ2 is adjusted to about 0.95-1.1 to stabilize the ethanol concentration of the cell culture to a concentration greater than 5 g/L, to about 5-17 g/L, to about 8-17 g/L, about 9-17 g/L, about 10-17 g/L, about 11-17 g/L, about 12-17 g/L about 8-16 g/L, about 8-15 g/L, about 8-14 g/L or about 8-13 g/L.

The present invention also provides a method for producing an antibody or antigen-binding fragment thereof in yeast comprising: a) providing a population of cultured Pichia pastoris cells, wherein each cell comprises a DNA segment encoding a heavy chain polypeptide and a light chain polypeptide of the antibody operably linked to a glyceraldehyde-3-phosphate (GAP) transcription promoter and a transcription terminator; b) culturing the cells of step (a) under batch fermentation conditions; c) culturing the cells of step (b) under fed-batch fermentation conditions comprising adjusting the first respiratory quotient (RQ1) about 1.1-1.6, to about 1.1-1.5, to about 1.2-1.6, to about 1.2-1.5, to about 1.3-1.4, or about 1.25-1.45 at about 20-40/48 hours of the fermentation process; d) harvesting the cells of step (c) at about 100-140 hours of the fermentation process; and e) recovering the antibody produced by the harvested cells of step (d). The yeast cells may, optionally, be of Pichia pastoris, Pichia methanolica, Pichia angusta, Pichia thermomethanolica or Saccharomyces cerevisiae. Optionally, RQ1 is adjusted to about 1.1-1.6 to increase the concentration of ethanol to about 15-23 g/L, about 17-23 g/L, about 17-22 g/L, about 18-22 g/L or about 19-21 g/L of the cell culture at about 40/48 hour of the fermentation process. Optionally, the method may further comprise a step of administering about 2.0-5.0 g/L of hydroxyurea to the cell culture at about 12-30 hours of the fermentation process. Optionally, the method may further comprise a step of administering about 2.0-4.5 g/L, about 2.0-4.0 g/L, about 3.0-4.0 g/L, about 2.5-5.0 g/L, about 2.1-2.9 g/L, about 2.2-2.8 g/L, about 2.6-2.8 g/L, about 2.5-2.8 g/L, about 2.6-2.9 g/L, about 2.3-2.7 g/L, about 2.4-2.6 g/L or about 2.5 of hydroxyurea is added at about 12-30 hours, 14-19 hours, 16-21 hours or about 16-22 hours of the fermentation process. Optionally, the method may further comprises a step of adjusting a second respiratory quotient (RQ2) to about 0.8-1.1, to about 0.8-1.15, to about 0.85-1.1, to about 0.85-1.15, to about 0.9-1.1, to about 0.9-1.15, to about 0.95-1.1, or to about 0.95-1.15 at about 40/48-100/140 hours of the fermentation process. The RQ2 may optionally be adjusted to about 0.95-1.1 to stabilize the ethanol concentration of the cell culture to a concentration greater than 5 g/L, to about 5-17 g/L, to about 8-17 g/L, about 9-17 g/L, about 10-17 g/L, about 11-17 g/L, about 12-17 g/L about 8-16 g/L, about 8-15 g/L, about 8-14 g/L or about 8-13 g/L. Optionally, the DNA segment encoding the heavy chain polypeptide and the light chain polypeptide are both operably linked to the same GAP promoter. Optionally, the DNA segment encoding the heavy chain polypeptide is operably linked to a first GAP promoter and the DNA segment encoding the light chain polypeptide is operably linked to a second GAP promoter. The GAP promoter may be derived from Pichia pastoris. The GAP promoter may have the nucleotide sequence of SEQ ID NO:20. The antibody or antigen-binding fragment thereof may specifically bind a cytokine (e.g., IL-6), receptor (e.g., chemokine receptor, cell surface receptor, interleukin receptor or a cytokine receptor) or a cell surface protein. Optionally, the antibody or antigen-binding fragment may be monoclonal or polyclonal. Optionally, the antibody or antigen-binding fragment may be multivalent, such as, for instance, a bispecific antibody. Optionally, the antibody may be a chimeric antibody, a human antibody or humanized antibody. Optionally, the antigen-binding fragment is Fab, Fab′, F(ab)₂, F(ab)₂, Fv or a single-chain Fv. Optionally, the antibody is an anti-human IL-6 monoclonal antibody, which may be a humanized anti-human IL-6 monoclonal antibody. The antibody may comprise a light chain polypeptide which comprises a light chain variable domain comprising the following CDRs: CDR1 having the amino acid sequence of SEQ ID NO:6; CDR2 having the amino acid sequence of SEQ ID NO:7; and CDR3 having the amino acid sequence of SEQ ID NO:8. The antibody may comprise a heavy chain polypeptide which comprises a heavy chain variable domain comprising the following CDRs: CDR1 having the amino acid sequence of SEQ ID NO:15; CDR2 having the amino acid sequence of SEQ ID NO:16; and CDR3 having the amino acid sequence of SEQ ID NO:17. Optionally, the antibody comprises a light chain polypeptide comprising a light chain variable domain comprising the following CDRs: CDR1 having the amino acid sequence of SEQ ID NO:6; CDR2 having the amino acid sequence of SEQ ID NO:7; and CDR3 having the amino acid sequence of SEQ ID NO:8; and a heavy chain polypeptide comprising a heavy chain variable domain comprising the following CDRs: CDR1 having the amino acid sequence of SEQ ID NO:15; CDR2 having the amino acid sequence of SEQ ID NO:16; and CDR3 having the amino acid sequence of SEQ ID NO:17. Optionally, the antibody may comprise a light chain variable domain comprising the amino acid sequence of SEQ ID NO:5. Optionally, the antibody may comprise a heavy chain variable domain comprising the amino acid sequence of SEQ ID NO:14. Optionally, the antibody comprises a light chain variable domain comprising the amino acid sequence of SEQ ID NO:5, and a heavy chain variable domain comprising the amino acid sequence of SEQ ID NO:14. The antibody may comprise or the antigen-binding fragment may further comprise a human heavy chain immunoglobulin constant domain of IgG, IgM, IgE or IgA, wherein the human IgG heavy chain immunoglobulin constant domain can be IgG1, IgG2, IgG3 or IgG4.

The present invention also provides a method for producing an antibody or antigen-binding fragment thereof in yeast comprising: a) providing a population of cultured Pichia pastoris cells, wherein each cell comprises a DNA segment encoding a heavy chain polypeptide and a light chain polypeptide of the antibody operably linked to a glyceraldehyde-3-phosphate (GAP) transcription promoter and a transcription terminator; b) culturing the cells of step (a) under batch fermentation conditions; c) culturing the cells of step (b) under fed-batch fermentation conditions comprising adjusting the respiratory quotient (RQ) to about 0.8-1.1, to about 0.8-1.15, to about 0.85-1.1, to about 0.85-1.15, to about 0.9-1.1, to about 0.9-1.15, to about 0.95-1.1, or to about 0.95-1.15 at about 40/48-100/140 hours of the fermentation process; d) harvesting the cells of step (c) at about 100-140 hours of the fermentation process; and e) recovering the antibody produced by the harvested cells of step (d). The RQ may optionally be adjusted to about 0.95-1.1 to stabilize the ethanol concentration of the cell culture to a concentration greater than 5 g/L, to about 5-17 g/L, to about 8-17 g/L, about 9-17 g/L, about 10-17 g/L, about 11-17 g/L, about 12-17 g/L about 8-16 g/L, about 8-15 g/L, about 8-14 g/L or about 8-13 g/L. The yeast cells may, optionally, be of Pichia pastoris, Pichia methanolica, Pichia angusta, Pichia thermomethanolica or Saccharomyces cerevisiae. Optionally, the DNA segment encoding the heavy chain polypeptide and the light chain polypeptide are both operably linked to the same GAP promoter. Optionally, the DNA segment encoding the heavy chain polypeptide is operably linked to a first GAP promoter and the DNA segment encoding the light chain polypeptide is operably linked to a second GAP promoter. The GAP promoter may be derived from Pichia pastoris. The GAP promoter may have the nucleotide sequence of SEQ ID NO:20. The antibody or antigen-binding fragment thereof may specifically bind a cytokine (e.g., IL-6), receptor (e.g., chemokine receptor, cell surface receptor, interleukin receptor or a cytokine receptor) or a cell surface protein. Optionally, the antibody or antigen-binding fragment may be monoclonal or polyclonal. Optionally, the antibody or antigen-binding fragment may be multivalent, such as, for instance, a bispecific antibody. Optionally, the antibody may be a chimeric antibody, a human antibody or humanized antibody. Optionally, the antigen-binding fragment is Fab, Fab′, F(ab)₂, F(ab)₂, Fv or a single-chain Fv. Optionally, the antibody is an anti-human IL-6 monoclonal antibody, which may be a humanized anti-human IL-6 monoclonal antibody. The antibody may comprise a light chain polypeptide which comprises a light chain variable domain comprising the following CDRs: CDR1 having the amino acid sequence of SEQ ID NO:6; CDR2 having the amino acid sequence of SEQ ID NO:7; and CDR3 having the amino acid sequence of SEQ ID NO:8. The antibody may comprise a heavy chain polypeptide which comprises a heavy chain variable domain comprising the following CDRs: CDR1 having the amino acid sequence of SEQ ID NO:15; CDR2 having the amino acid sequence of SEQ ID NO:16; and CDR3 having the amino acid sequence of SEQ ID NO:17. Optionally, the antibody comprises a light chain polypeptide comprising a light chain variable domain comprising the following CDRs: CDR1 having the amino acid sequence of SEQ ID NO:6; CDR2 having the amino acid sequence of SEQ ID NO:7; and CDR3 having the amino acid sequence of SEQ ID NO:8; and a heavy chain polypeptide comprising a heavy chain variable domain comprising the following CDRs: CDR1 having the amino acid sequence of SEQ ID NO:15; CDR2 having the amino acid sequence of SEQ ID NO:16; and CDR3 having the amino acid sequence of SEQ ID NO:17. Optionally, the antibody may comprise a light chain variable domain comprising the amino acid sequence of SEQ ID NO:5. Optionally, the antibody may comprise a heavy chain variable domain comprising the amino acid sequence of SEQ ID NO:14. Optionally, the antibody comprises a light chain variable domain comprising the amino acid sequence of SEQ ID NO:5, and a heavy chain variable domain comprising the amino acid sequence of SEQ ID NO:14. The antibody may comprise or the antigen-binding fragment may further comprise a human heavy chain immunoglobulin constant domain of IgG, IgM, IgE or IgA, wherein the human IgG heavy chain immunoglobulin constant domain can be IgG1, IgG2, IgG3 or IgG4. Optionally, the heavy chain polypeptide of the produced antibody has an apparent molecular weight of about 49 kD as determined on a reducing SDS-polyacrylamide gel. Optionally, the heavy chain polypeptide of the produced antibody is substantially free of cleavage, wherein cleavage of the heavy chain polypeptide results in an about 37 kD band and an about 19 kD band on a reducing SDS-PAGE gel.

The present invention also provides a method for producing an antibody or antigen-binding fragment thereof in Pichia pastoris substantially free of cleavage comprising a) providing a population of cultured Pichia pastoris cells, wherein each cell comprises a DNA segment encoding a heavy chain polypeptide and a light chain polypeptide of the antibody operably linked to a glyceraldehyde-3-phosphate (GAP) transcription promoter and a transcription terminator; b) culturing the cells of step (a) under batch fermentation conditions; c) culturing the cells of step (b) under fed-batch fermentation conditions comprising adjusting the respiratory quotient (RQ) to about 0.8-1.1, to about 0.8-1.15, to about 0.85-1.1, to about 0.85-1.15, to about 0.9-1.1, to about 0.9-1.15, to about 0.95-1.1, or to about 0.95-1.15 at about 40/48-100/140 hours of the fermentation process; d) harvesting the cells of step (c) at about 100-140 hours of the fermentation process; and e) recovering the antibody produced by the harvested cells of step (d); and wherein the heavy chain polypeptide of the produced antibody is substantially free of cleavage, and wherein cleavage of the heavy chain polypeptide results in an about 37 kD band and an about 19 kD band on a reducing SDS-PAGE gel. Optionally, the antibody is substantially free of cleavage if less than one percent of the heavy chain polypeptide is cleaved as determined on a reducing SDS-PAGE gel.

The present invention also provides a method for producing an antibody or antigen-binding fragment thereof in Pichia pastoris comprising: a) providing a population of cultured Pichia pastoris cells, wherein each cell comprises a DNA segment encoding a heavy chain polypeptide and a light chain polypeptide of the antibody operably linked to a promoter and a transcription terminator; b) culturing the cells of step (a) under batch fermentation conditions; c) culturing the cells of step (b) under fed-batch fermentation conditions comprising administering about 2.0-5.0 g/L of hydroxyurea to the cell culture at about 12-30 hours of the fermentation process; d) harvesting the cells of step (c) at about 100-140 hours of the fermentation process; and e) recovering the antibody produced by the harvested cells of step (d). Optionally, the promoter is a glyceraldehyde-3-phosphate (GAP) promoter, such as the nucleotides of SEQ ID NO:20. Optionally, the DNA segment encoding the heavy chain polypeptide and the light chain polypeptide are both operably linked to the same GAP promoter. Optionally, the DNA segment encoding the heavy chain polypeptide is operably linked to a first GAP promoter and the DNA segment encoding the light chain polypeptide is operably linked to a second GAP promoter. The GAP promoter may be derived from, for example, Pichia pastoris, Pichia methanolica, Pichia angusta or Pichia thermomethanolica. Optionally, the antibody is an anti-human IL-6 antibody. Optionally, the light chain polypeptide of the anti-human IL-6 antibody comprises the following CDRs: CDR1 having the amino acid sequence of SEQ ID NO:6; CDR2 having the amino acid sequence of SEQ ID NO:7; and CDR3 having the amino acid sequence of SEQ ID NO:8. Optionally, the heavy chain polypeptide of the anti-human IL-6 antibody comprises the following CDRs: CDR1 having the amino acid sequence of SEQ ID NO:15; CDR2 having the amino acid sequence of SEQ ID NO:16; and CDR3 having the amino acid sequence of SEQ ID NO:17. Optionally, the anti-human IL-6 antibody comprises a light chain polypeptide comprising a light chain variable domain comprising the following CDRs: CDR1 having the amino acid sequence of SEQ ID NO:6; CDR2 having the amino acid sequence of SEQ ID NO:7; and CDR3 having the amino acid sequence of SEQ ID NO:8; and a heavy chain polypeptide comprising a heavy chain variable domain comprising the following CDRs: CDR1 having the amino acid sequence of SEQ ID NO:15; CDR2 having the amino acid sequence of SEQ ID NO:16; and CDR3 having the amino acid sequence of SEQ ID NO:17. Optionally, the light chain variable domain of the anti-human IL-6 antibody comprises the amino acid sequence of SEQ ID NO:5. Optionally, the heavy chain variable domain of the anti-human IL-6 antibody comprises the amino acid sequence of SEQ ID NO:14. The antibody or antigen-binding fragment, such as an antibody or antigen-binding fragment that specifically binds to a lymphocyte antigen, cytokine, cytokine receptor, growth factor, growth factor receptor, interleukin, interleukin receptor or any combination thereof, is human, humanized or chimeric. The antibody may comprise a human heavy chain immunoglobulin constant domain of IgG, IgM, IgE or IgA. The human IgG heavy domain immunoglobulin constant domain may be IgG1, IgG2, IgG3 or IgG4. The antigen-binding fragment may further comprise a human heavy chain immunoglobulin constant domain of IgG, IgM, IgE or IgA, which the IgG domain can be IgG1, IgG2, IgG3 or IgG4. The antibody or antigen-binding fragment may be multivalent, such as bispecific, trispecific or tetraspecific. The amount of hydroxyurea added at about 12-30 hours, at about 16-22 hours, at about 14-19 hours, or at about 16-21 hours of the fermentation process may be about 2.0-4.5 g/L, about 2.0-4.0 g/L, about 3.0-4.0 g/L, about 2.5-5.0 g/L, about 2.1-2.9 g/L, about 2.2-2.8 g/L, about 2.6-2.8 g/L, about 2.5-2.8 g/L, about 2.6-2.9 g/L, about 2.3-2.7 g/L, about 2.4-2.6 g/L or about 2.5 g/L. The method may further comprise a step of adjusting a first respiratory quotient (RQ1) to about 1.36-1.6, to about 1.36-1.45, to about 1.45-1.6, or to about 1.4-1.5 at about 16/21-32/48 hours of the fermentation process. The method may also further comprise the step of increasing the concentration of ethanol to about 18-22 g/L or about 19-21 g/L of the cell culture at about 16/21-32/48 hours of the fermentation process, which may include maintaining this ethanol concentration for a period of up to about 8 hours, up to about 7 hours, up to about 6 hours, up to about 5 hours, up to about 4 hours, up to about 3 hours, up to about 2 hours, up to about 1 hour, up to about 30 minutes or up to about 1 second. The method may further comprise a step of adjusting a second respiratory quotient (RQ2) to about 0.8-1.06, to about 0.85-1.06, to about 0.90-1.06, to about 0.95-1.06 or less than 1.07 at about 32/48-100/140 hours of the fermentation process. The method may further comprise a step of stabilizing the ethanol concentration of the cell culture to a concentration greater than 5 g/L, to about 5-17 g/L, to about 8-17 g/L, about 9-17 g/L, about 10-17 g/L, about 11-17 g/L, about 12-17 g/L about 8-16 g/L, about 8-15 g/L, about 8-14 g/L or about 8-13 g/L at about 32/48-100/140 hours of the fermentation process.

The novel fermentation process uses unique methods for hydroxyurea application, and/or ethanol control, and/or RQ control in, for example, Pichia pastoris (P. pastoris) fermentation for production of an antibody or antigen-binding fragment thereof. The methodology differs from the conventional methods in at least four aspects. First, a strategy comprised of using two RQ control regimes and hydroxyurea to achieve unique ethanol and cell density profiles. The process was initiated as a conventional P. pastoris fermentation process by approximately 20 hours run time. The addition of hydroxyurea and the first RQ control regime at set point of about 1.2-1.6 (optionally about 1.3-1.5) were then applied to slow down cell growth and achieve accumulation of ethanol to about 18-22 grams/Liter (g/L) at about 40 hours run time. Reduced fed-batch rate and the second RQ control regime at set point of about 0.80-1.07 (optionally about 1.00-1.06) were applied afterwards to achieve a steady state of both ethanol and cell density. Antibody production was enhanced under these conditions. Second, unlike the hydroxyurea dose used to inhibit cell division (˜5.7 g/L) in the literature, the present invention uses a much lower dose of hydroxyurea (about 2.0-5.0 g/L). At reduced hydroxyurea concentration, cell division may not be inhibited, which is evidenced by the increased wet cell weight as compared to the control. Correspondingly, integrated wet cell weight was increased that led to an increase in antibody production. Third, the ethanol level was allowed to reach a peak of 18-22 g/L, which is higher than the common recommendation in the art (e.g., ˜1.0% v/v, or 7.6 g/L). Finally, the second RQ control regime contributes not only to the ethanol and biomass profiles, but also to an increase in product quality in terms of avoiding a clip on the heavy chain of the antibody.

The fermentation process of the present invention encompasses at least one of the steps of a three step process including two seed culture steps and one main culture step. The Seed II culture step can be performed in either shake flasks or a fermentor. The seed cultures follow the traditional yeast batch mode fermentation, while the fermentation process at the main culture step is comprised of the unique ethanol control strategy to balance cell growth and specific antibody production rate, and/or addition of hydroxyurea to enhance antibody productivity by increasing integrated wet cell weight, and/or a RQ control strategy to maintain optimum ethanol profile and improve product quality.

The novel fermentation process for the production of an antibody or antigen-binding fragment thereof by fermentation (e.g., fed-batch fermentation) of, for example, P. pastoris. One aspect of the process includes a strategy of two RQ control regimes to achieve unique ethanol and cell density profiles. After a conventional fed-batch mode of fermentation for approximately 16-22 hours run time, hydroxyurea at about 2.0-5.0 g/L was added to the fermentation culture to control cell growth and help sustain a constant cell density. The first RQ control regime at set point of about 1.2-1.6 (optionally about 1.3-1.5) was then applied to slow down cell growth and achieve accumulation of ethanol to about 18-22 g/L by about 40 hour run time. Reduced fed rate and the second RQ control regime at set point of about 0.80-1.07 (optionally about 1.00-1.06) was then applied to achieve a steady state of both ethanol and cell density. In addition, the method of the second RQ control regime at set point of about 0.80-1.07 also eliminated an about 37 kD/19 kD clipping variant of the antibody. The fermentation process that includes, but is not limited to, the above methods achieved >100% productivity enhancement in the production of a humanized anti-IL-6 antibody.

### Fermentation Media

Seed Medium is described below in Table 1.

TABLE 1
Seed medium

| Ingredient¹ | Concentration | |
|---|---|---|
| Yeast extract | 23-25 | g/L |
| KH₂PO₄ | 9.0-10.0 | g/L |
| K₂HPO₄ | 1.8-1.9 | g/L |
| Glucose | 19-21 | g/L |
| Yeast nitrogen base w/o amino acids | 13-14 | g/L |
| D-Biotin | 0.3 8-0.42 | mg/L |

¹Keeping the same molarity, any chemical (X nH₂O, n >= 0) can be replaced by another chemical containing the same activated ingredient but various amount of water (X kH₂O, k ≠ n).

Trace element solution is described below in Table 2.

TABLE 2
Trace element solution

| Ingredient¹ | Concentration | |
|---|---|---|
| CuSO₄ 5H₂O | 5.7-6.3 | g/L |
| Sodium iodide | 0.076-0.084 | g/L |
| MnSO₄ H₂O | 2.8-3.2 | g/L |
| Sodium molybdate 2H₂O | 0.19-0.21 | g/L |
| H₃BO₃ | 0.019-0.021 | g/L |
| CoCl₂ 6H₂O | 0.47-0.53 | g/L |
| ZnCl₂ | 19-21 | g/L |
| FeSO₄ 7H₂O | 62-68 | g/L |
| Biotin | 0.19-0.21 | g/L |
| Sulfuric Acid | 4.8-5.2 | ml/L |

¹Keeping the same molarity, any ingredient (X nH₂O, n >= 0) can be replaced by another ingredient containing the same activated chemical but various amount of water (X kH₂O, k ≠ n).

Batch Medium is described below in Table 3.

TABLE 3
Batch medium

| Ingredient¹ | Concentration | |
|---|---|---|
| KH₂PO₄ | 2.1-2.4 | g/L |
| K₂HPO₄ | 0.41-0.45 | g/L |
| (NH₄)SO₄ | 9.2-10.2 | g/L |
| YE | 25-28 | g/L |
| AF | 1.4-1.6 | g/L |
| PTM1c | 3.7-4.1 | mL/L |
| Glucose H₂O | 33-37 | g/L |
| MgSO₄ 7H₂O | 2.4-2.8 | g/L |

¹Keeping the same molarity, any chemical (X nH₂O, n >= 0) can be replaced by another chemical containing the same activated ingredient but various amount of water (X kH₂O, k ≠ n).

Feed Medium is described below in Table 4. Optionally, the Feed Medium may be a mixture of Glucose Feed Medium and Yeast Extract Feed Medium. In this case, the fed rates were adjusted to deliver the equivalent dose of each ingredient.

TABLE 4
Feed medium

| Ingredient¹ | Concentration | |
|---|---|---|
| Glucose | 470-530 | g/L |
| MgSO₄ 7H₂O | 2.8-3.2 | g/L |
| Yeast Extract | 47-53 | g/L |
| Antifoam | 0.4-0.6 | g/L |
| PTM1c | 7-13 | mL/L |

¹Keeping the same molarity, any chemical (X nH₂O, n >= 0) can be replaced by another chemical containing the same activated ingredient but various amount of water (X kH₂O, k ≠ n).

Hydroxyurea solution is described below in Table 5.

TABLE 5
Hydroxyurea solution

| Ingredient¹ | Concentration | |
|---|---|---|
| Hydroxyurea | 75-90 | g/L |
| EtOH | 75-90 | mL/L |

### Fermentation Process

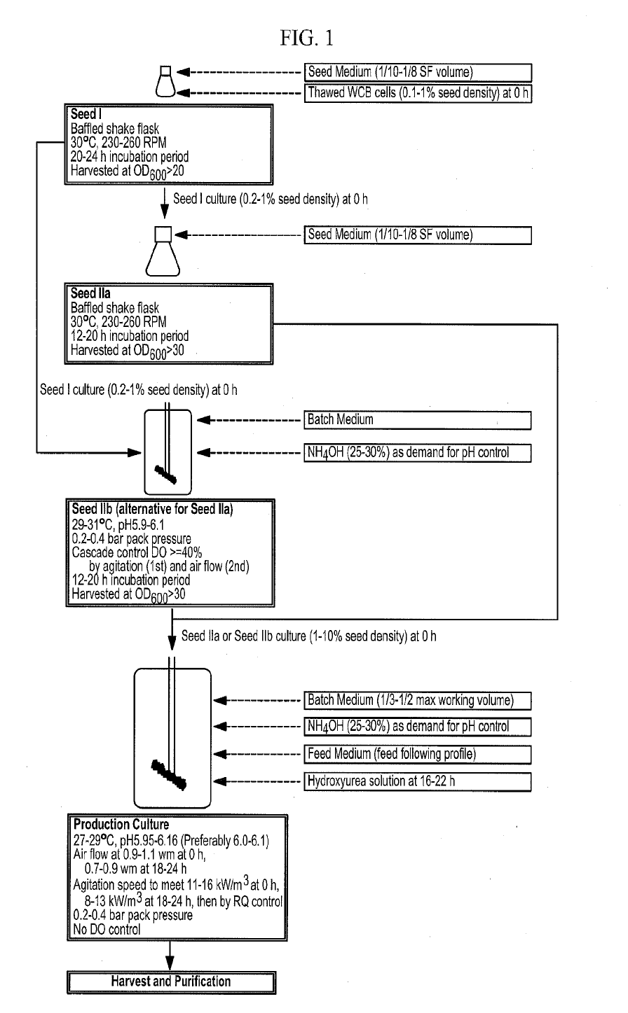

The fermentation process for the production of antibodies or antigen-binding fragments thereof is shown in FIG. 1. The antibody is produced by yeast fermentation, such as in P. pastoris. The fermentation is initiated, for example, from the thawing of a frozen vial of a cell bank. The thawed cells are then propagated two passages in shake flasks as the Seed I and Seed II cultures, respectively. Optionally, Seed II can be performed in a bioreactor. Finally, the main culture is inoculated with Seed II culture and operated as a fed-batch mode of fermentation for the production of the antibody.

1. Seed I Step

Thawed cells of the cell bank are transferred to a baffled shake flask (1 to 4 baffles) containing seed medium of 10-20% of flask working volume as the Seed I culture. The seed density is usually 0.1 to 1.0%. The Seed I culture is incubated at 29-31° C. and 220-260 RPM. The culture is harvested once reaching optical density at about 600 nm (OD₆₀₀) of 15-30 (optionally 20-30). This step usually lasts 20-26 hours (optionally 23-25 hours).

2. Seed II Step

The harvested Seed I culture is inoculated to a baffled shake flask (1 to 4 baffles) containing seed medium of 10-20% of flask working volume as the Seed II culture. The seed density is adjusted to meet post-inoculation OD₆₀₀ of 0.1-1.0 (optionally 0.4-0.6). The Seed II culture is then incubated at 29-31° C. and 220-260 RPM. The culture is harvested once reaching OD₆₀₀ of about 20-50 (optionally 30-40). This step usually lasts about 12-20 hours (optionally about 14-18 h). Optionally, Seed II can be performed in a bioreactor using the Batch Medium containing reduced antifoam concentration as described, for example, in FIG. 1.

3. Main Culture Step

The main culture is initiated from inoculation with Seed II culture and ended with harvest for downstream processing, which comprises the following two phases.

3.1. Batch Culture Phase

The batch culture phase is initiated from inoculation of the main culture and ended with depletion of glucose. The harvested Seed II culture is inoculated to a bioreactor containing batch medium of 30-40% of maximum working volume. The seed density is about 1-10% (optionally about 2-5%) of initial working volume post-inoculation. The initial engineering parameters are set, for example, as follows:
Temperature: 27-29° C.;
Agitation (P/V): 10-16 KW/m³;
Headspace pressure: 0.2-0.4 Bar;
Bottom air flow: 0.9-1.1 VVM;
DO: no control;
pH: 6.00˜6.10 controlled by 24-30% NH₄OH.

The agitation (revolutions per minute or rpm) and airflow (standard liters per minute or slpm) to meet the initial P/V and VVM specifications are kept constant during this phase. The other engineering parameters are also kept constant. Batch culture phase ends and the feed culture phase begins when glucose is depleted, which is indicated by dissolved oxygen (DO) spike (DO value increases by >30% within a few minutes). Batch culture phase usually lasts about 10-15 hours (optionally about 11-13 hours).

3.2 Fed-Batch Culture Phase

The fed-batch culture phase covers from feed start when glucose is depleted to the end of fermentation. This phase can be further divided into three periods, namely cell mass buildup, ethanol buildup, and ethanol stabilization periods. The production of the antibody occurs in the last two periods.

3.2.1 Cell Mass Buildup Period

The cell mass buildup phase is initiated from feed start when glucose is depleted. The feed rate of the feed medium is based on glucose, which is about 10-12 grams glucose per liter of initial volume per hour (g/L/h). The engineering parameters are kept the same as the batch culture phase. Hydroxyurea is added at about 5-8 hours post feeding to stabilize cell density at 350-450 g/L wet cell weight. The hydroxyurea dose may be added to a concentration of about 2.0-5.0 gram per liter (g/L), optionally about 2.0-3.0 g/L, of initial working volume. The culture is switched to the next period 2 hours later at about 16-21 hours run time. Thus, the cell mass buildup period is from about 10/15 hours to about 16/21 hours of the fermentation process. The cell mass buildup period can be from about 10 hours to about 21 hours of the fermentation process, from about 10 hours to about 16 hours of the fermentation process, from about 15 hours to about 21 hours of the fermentation process or about 15 hours to about 16 hours of the fermentation process.

3.2.2 Ethanol Buildup Period

The ethanol buildup phase starts about 2 hours post hydroxyurea addition.

Agitation and airflow are then reduced to 75-85% of original level and the RQ value record is started. Agitation is further adjusted to keep the RQ value at about 1.2-1.6 (optionally about 1.3-1.5), that enables accumulation of ethanol to peak of about 15-23 g/L (optionally about 18-22 g/L) at about 32-48 hours run time when the culture is shifted to the next period. Thus, the ethanol buildup period is from about 16/21 hours to about 32/48 hours of the fermentation process. The ethanol buildup period can be from about 16 hours to about 32 hours of the fermentation process, from about 16 hours to about 48 hours of the fermentation process, from about 21 hours to about 32 hours of the fermentation process or about 21 hours to about 48 hours of the fermentation process.

3.2.3 Ethanol Stabilization Period

The ethanol stabilization period is initiated by reducing feed to 50% of its original rate. Agitation is further adjusted to maintain RQ value of about 0.95-1.1 (optionally below about 1.07). The feeding rate is increased by 5% of the current value every other 12 hours. The RQ value allows a steady state of ethanol metabolism. As a result of the dilution factor caused by feeding, the ethanol concentration of the fermentation broth is slowly declining until harvest, where the concentration is usually greater than 5 g/L. The ethanol stabilization period is from about 32/48 hours to about 100/140 hours of the fermentation process. The ethanol stabilization period can be from about 32 hours to about 100 hours of the fermentation process, from about 32 hours to about 140 hours of the fermentation process, from about 48 hours to about 100 hours of the fermentation process or about 48 hours to about 140 hours of the fermentation process.

### Downstream Purification and Analytical Methods

A conventional purification process (Forss, A. et al., BioProcess International, 9:64-68 (2011)) was used for downstream purification. The glucose and ethanol were measured by YSI 2700 (YSI Incorporated, Yellow Springs, Ohio), O₂ and CO₂ of the exhaust line were measured by Questor GP Process Mass Spectrometer (ABB Extrel, Pittsburgh, Pa.) and the RQ value was calculated using below Equation [1]. The wet cell weight (WCW) was measured by centrifuging one (1) milliliter (mL) fermentation broth at 13,200 rpm for 10 minutes, weighing pellet, and calculated ratio of pellets weight (g) over volume (mL). The supernatant titer (g/L) was measured by the HPLC method and the whole broth (WB) titer was then calculated by below Equation[2]. Non reduced and reducing SDS-PAGE gels are performed following standard method. The about 37 kD and about 19 kD bands visible on reducing SDS-PAGE gel were characterized by protein sequencing.

RQ=0.79*%_{CO}_{₂}/(21−0.21*%_{CO}_{₂}−%_{O}_{₂}) [1]

WB_Titer=Supernatant_Titer*(1−WCW/1000) [2]

The invention is further illustrated by the following non-limiting examples.

### EXAMPLES

### Example 1

### Effects of Ethanol on Cell Growth and Antibody Production in P. pastoris Fermentation

Example 1 demonstrates the effects of residual ethanol concentration on cell growth and productivity of an anti-IL-6 humanized monoclonal antibody. The novel fermentation process described herein was used to produce a humanized anti-IL-6 monoclonal antibody having the light and heavy chain polypeptide sequences of SEQ ID NOs:3 and 12, respectively. The media and processes of Seed I and Seed II cultures are described herein. The main culture process was also followed as described herein, except for the following three differences. First, hydroxyurea was not yet applied. Second, RQ control was also not yet applied. Third, five ethanol levels were established during the fed-batch culture phase in duplicate lots by adjusting agitation.

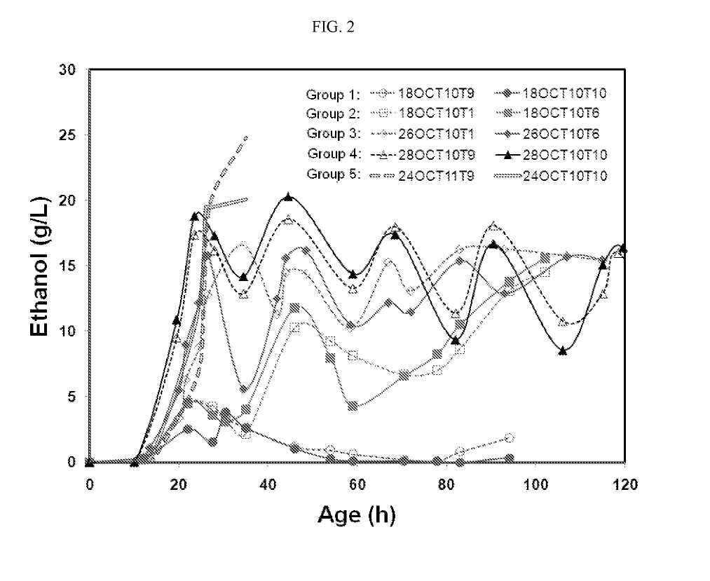

As shown in FIG. 2, five distinct ethanol levels were observed in ten fermentation lots, which were the basis for grouping fermentation lots. Group 1 (lots 18OCT10T9 and T10) had 3-5 g/L ethanol at 20-30 hours run time and maintained 0-5 g/L ethanol afterwards. Group 2 (lots 18OCT10T1 and T6) also had 3-5 g/L ethanol at 20-30 hours run time but reached 10-12 g/L ethanol at 40-45 hours run time and then maintained 5-15 g/L ethanol afterwards. Group 3 (Lots 26OCT10T1 and T6) reached 14-16 g/L ethanol for a short period (<3 h) at 20-30 hours and 40-45 hours run time, respectively, and then maintained 10-16 g/L ethanol afterwards. Group 4 (lots 28OCT10T9 and T10) reached ethanol level of 17-20 g/L for a short period (<3 hours) at 20-30 hours and 40-45 hours run time, respectively, and then maintained 8-17 g/L ethanol afterwards. Group 5 (lots 24OCT10T9 and T10) reached ethanol level greater than 20 g/L for more than 8 hours after 20 hours run time.

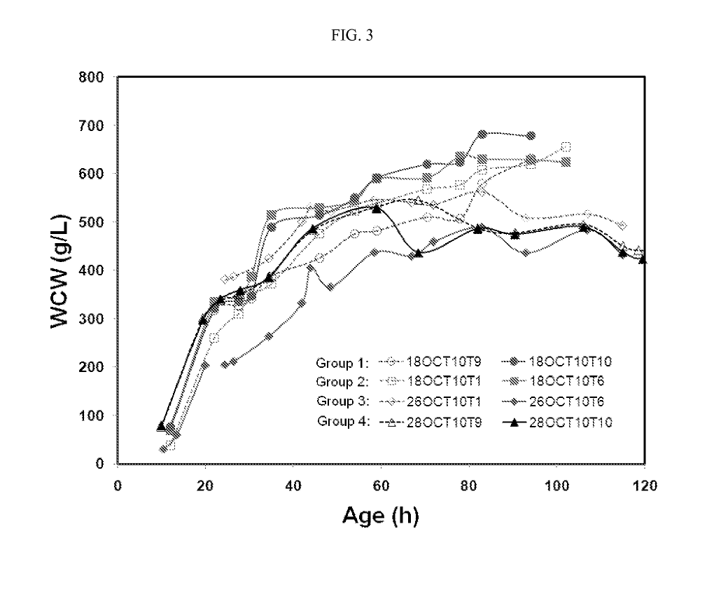

Wet cell weight (WCW) profiles are shown in FIG. 3, except for Group 5 (lots 240CT10T9 and T10) which was terminated early due to cell death caused by exposing high ethanol level (>20 g/L) for 8 hours. Groups 1 and 2 demonstrated that the cultures were able to increase cell density and were able to reach >600 g/L WCW by 80 hours run time at the low ethanol level (<13 g/L). Groups 3 and 4 demonstrated that one or two periods of high ethanol concentration (14-20 g/L for <3 hours in this instance) between 20-50 hours could lead to a relative constant WCW level below 500 g/L afterwards.

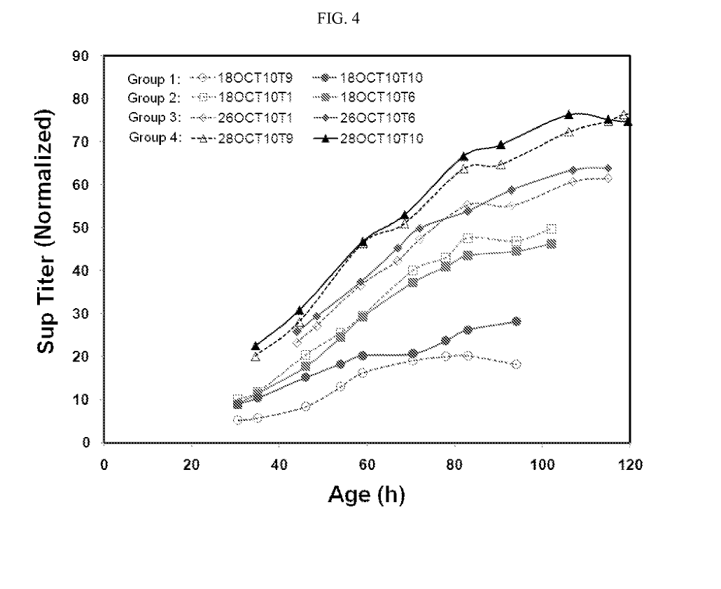

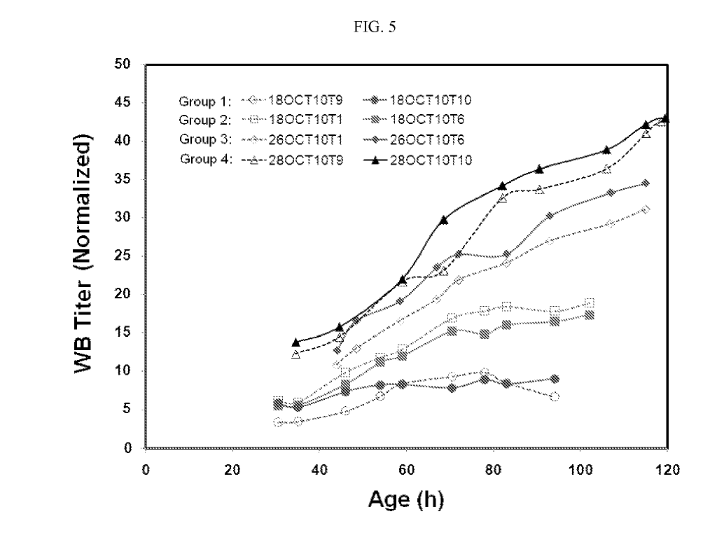

The supernatant and whole broth titers of Group 1 through Group 4 are shown in FIG. 4 and FIG. 5, respectively. The trend of increased titers was observed with the increased peak ethanol level in the period between 20 and 50 hours run time. The highest titers were seen in Group 4 that reached peak ethanol level of 18.5-21 g/L at 40-48 hours and then maintained an ethanol level between 8-17 g/L for the remaining period of fermentation. The “baseline” productivity is represented in Group 2. The Group 2 standard ethanol control strategy maintained the ethanol level at ˜10 g/L until 83 hours run time of the fermentation process. This group produced WB titer of 16.1 and 18.5 normalized units at 83 hours run time. Group 4, however, produced WB titer of 32.7 and 34.3 normalized units at 82 hours. Therefore, a 94% productivity improvement was achieved by using the conditions of Group 4 as compared to Group 2.

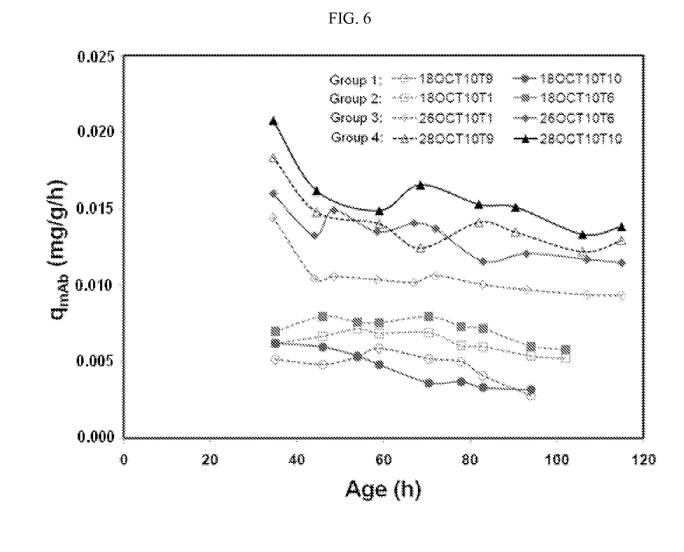

The anti-IL-6 antibody production rates of Group 1 through Group 4 are presented in FIG. 6, which were based on the units (milligrams or mg) of the antibody produced from one unit (g) of wet cell weight per hour (h). The trend of increased production rates was observed with the increased peak ethanol level in the period between 20 and 50 hours run time. The highest production rates were again seen in Group 4, which was consistent with the titer results described in the preceding paragraph.

In summary, Example 1 demonstrated the impact of ethanol concentration on cell growth and on antibody production rate. Based on the results of Group 4 (lots 280CT10T9 and T10), a fermentation process with 4-step monitoring of ethanol and cell density was recommended as the new fermentation process. The first period covers 0 to ˜12 hours run time, which is in a conventional batch culture phase. The subsequent three periods are in the fed-batch culture phase. The second period covers ˜12 to ˜20 hours run time, which focuses on cell mass build up with minimum ethanol accumulation (<13 g/L, optionally <10 g/L). The third period covers ˜20 hours to 40-48 hours run time, which focus of ethanol build up to the peak of 17-22 g/L. The last period covers remaining fermentation period until harvest, in which the ethanol level was maintained at 8-17 g/L with relative constant wet cell weight at ˜400 g/L. This new fermentation process (Group 4) showed 94% productivity improvement as compared to the previous conventional standard (Group 2). This new fermentation process as exemplified in Group 4 was further developed in Examples 2-4.

### Example 2

### Effects of Hydroxyurea on Cell Growth and Protein Productivity in P. pastoris Fermentation

Example 2 demonstrates the effects of hydroxyurea on cell growth and productivity of the anti-IL-6 antibody in Run 01MAY11. The media and the Seed I and Seed II processes are as described herein. At the main culture step, the control cultures were operated to have ethanol profiles mimicking the new fermentation process (Group 4 process in Example 1) as demonstrated by Lots 28OCT10T9 and T10. The treatment cultures were operated the same way plus adding hydroxyurea 5 hours after feed start. The amount of hydroxyurea added was to bring the residual hydroxyurea concentration of fermentation broth to 2.6-2.8 g/L based on initial working volume. The control and the treatment were run in triplicate bioreactors (Sartorius BIOSTAT® C).

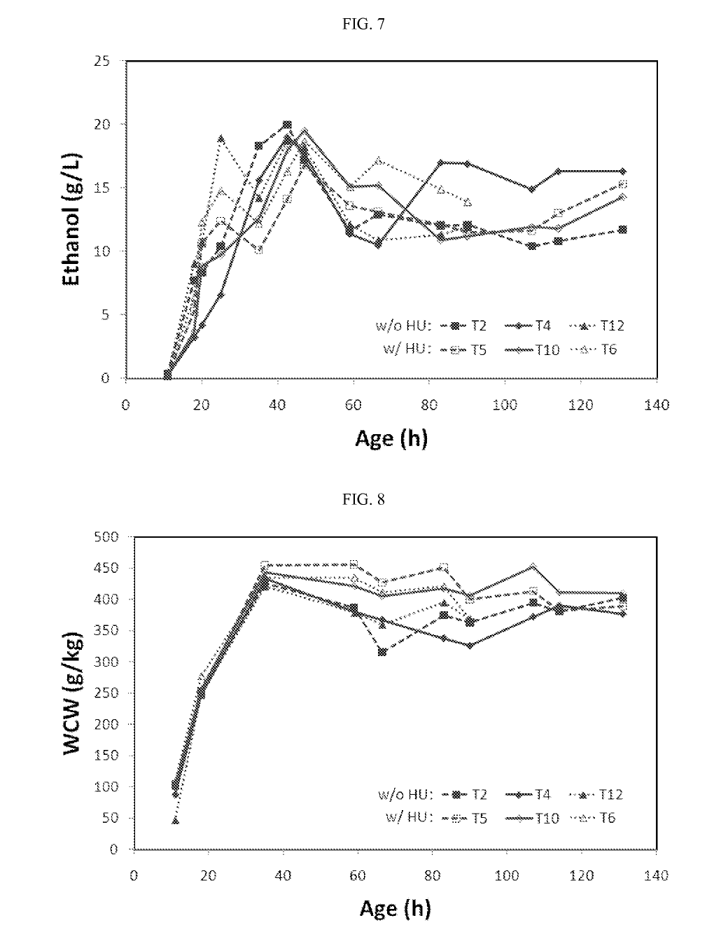

The ethanol and wet cell weight (WCW) profiles are presented in FIG. 7 and FIG. 8. To simplify the new fermentation process described above in Example 1, it was designed to increase the ethanol concentration to 17-20 g/L ethanol at ˜45 hours and then maintain an ethanol level of 10-17 g/L until the end of fermentation. The ethanol concentration aims to force cell metabolism shift to the steady status of cell growth and ethanol production. FIG. 7 demonstrated that all cultures except for T12 received ethanol concentrations at 17-20 g/L once at ˜45 hours, while T12 culture twice received high ethanol concentrations at 25 hours and 45 hours run time, respectively. FIG. 7 and FIG. 8 also showed that ethanol level and wet cell weight were maintained relative steady after the high ethanol concentration at ˜45 hours run time. Even though the ethanol level of T4 culture was turbulent between 45 hours and 80 hours due to the effect of impeller engagement and engineering parameter adjustment, culture was able to maintain the relative steady ethanol level afterwards. FIG. 8 showed that all cultures reached peak cell density at 30-40 hours and then maintained at steady value to the end of fermentation. However, the hydroxyurea treatment lots reached higher WCW (˜450 g/kg) than the control lots (˜400 g/Kg). This result differs from the observed reports in the literature (Doran, P. M. et al., Biotechnol. Bioeng., 28:1814-1831 (1986)), of which cell mass was reduced by 50% after addition of 5.7 g/L hydroxyurea into the suspended S. cerevisiae cells. The reduction of cell mass was contributed by inhibiting cell division. In our case, we observed an increasing, rather than reducing, cell mass in the hydroxyurea treatment lots, which might reflect to the dose response of hydroxyurea. We used 50% of the hydroxyurea dose (2.6-2.8 g/L) as compared to the dose reported in the literature (Doran, P. M. et al., Biotechnol. Bioeng., 28:1814-1831 (1986)), which, while not wishing to be bound by any particular theory, might not be strong enough to inhibit cell division but may assist the cells in increasing their tolerance to the high ethanol concentration and as a result gain more cell mass after hydroxyurea treatment.

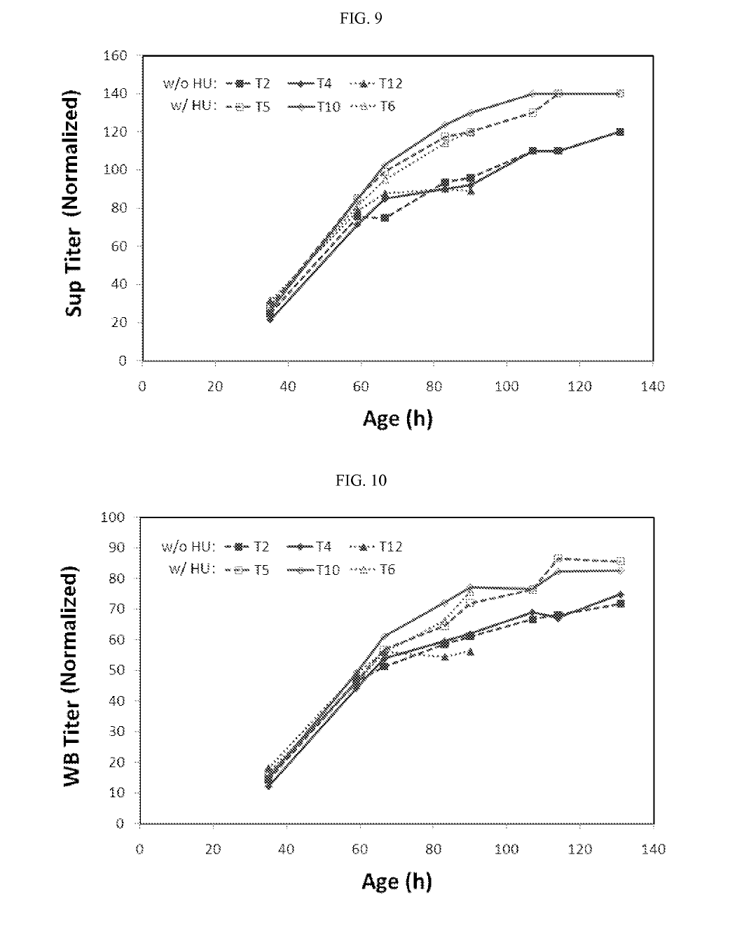

The profiles of supernatant and whole broth titers are presented in FIG. 9 and FIG. 10, respectively. The difference in supernatant and whole broth titers became significant after 60 hours fermentation. Including all triplicate data, three hydroxyurea treatment lots produced 75 normalized units, while three control lots produced 59.8 normalized units of average whole broth titer at 90 hours run time, indicating 25% productivity improvement by hydroxyurea treatment.

TABLE 6
Effects of Hydroxyurea on Cell Growth and Antibody
Productivity of Fermentation Run 01 MAY 2011

| | | | |
|---|---|---|---|
| | WCW | Sup Titer | WB Titer |
| Age (h) | (g/kg) | (Normalized) | (Normalized) |

Lots T2, T4 andT12 − Hydroxyurea, the control

| | | | |
|---|---|---|---|
| 59 | 382 ± 4 | 75.3 ± 3.6 | 46.6 ± 2.3 |
| 90 | 352 ± 23 | 92.3 ± 3.5 | 59.8 ± 3.1 |

Lots T5, T6 andT10 + Hydroxyurea

| | | | |
|---|---|---|---|
| 59 | 437 ± 17 | 83.9 ± 2.2 | 47.2 ± 1.8 |
| 90 | 392 ± 20 | 123.3 ± 5.8 | 75.0 ± 2.7 |

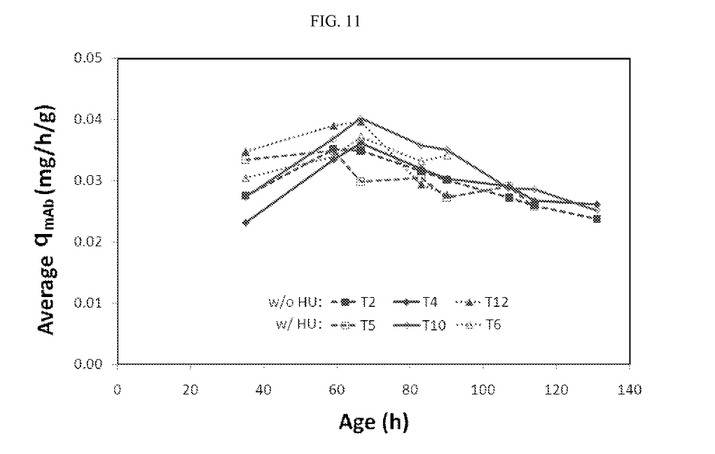

The average specific antibody production rates (in wet cell weight basis) were then calculated. As shown in FIG. 11, the profiles of specific production rate of the treatment and the control lots are overlapped. After noting the WCW profile demonstrated in FIG. 8 that the hydroxyurea treatment maintained higher WCW (˜450 g/kg) than the control lots (˜400 g/Kg) after a high ethanol concentration at 17-22 g/L ethanol, it can be reasonably concluded that the enhanced whole broth titer after 60 hours run time as shown in FIG. 10 and Table 6 was caused by the increased cell mass.

In summary, Example 2 demonstrated that the addition of 2.6-2.8 g/L hydroxyurea at about 5 hours after feed start would enhance productivity of the antibody. Without wishing to be bound by a particular theory, the hydroxyurea treatment may help cells to increase tolerance to a high ethanol concentration and hence gain more cell mass during ethanol build up and after the high ethanol concentration. Approximately 25% productivity improvement was achieved by this hydroxyurea treatment. Without wishing to be bound by a particular theory, the enhanced antibody productivity may have benefited by the increase in cell mass.

### Example 3

### Effect of RQ Control on Product Purity of P. pastoris Fermentation: Case 1

Example 3 demonstrates the effects of respiratory quotient (RQ) control on product quality of the humanized anti-IL-6 antibody based on the data described herein. Specifically, the desired antibody quality is the 37/19 kD clipped variant below detectable level (<=1% of the antibody). The anti-IL-6 antibody 37/19 kD clipped variant is the result of a clip on the heavy chain and can be visible on a reducing SDS-PAGE gel. The media and process are described herein. In the period between May, 2011 and August, 2011, the RQ control strategies were tested to keep the ethanol profiles described in Example 1, of which the culture's ethanol level reached its peak of 17-20 g/L at ˜45 hours run time to give the cells a high ethanol concentration and maintain the ethanol level at 10-17 g/L thereafter.

Except for Run 19JUN11 which was a side-by-side comparison experiment for RQ control criterion evaluation and is described in Example 4, the retrospective data of five lots were analyzed in this Example 3.

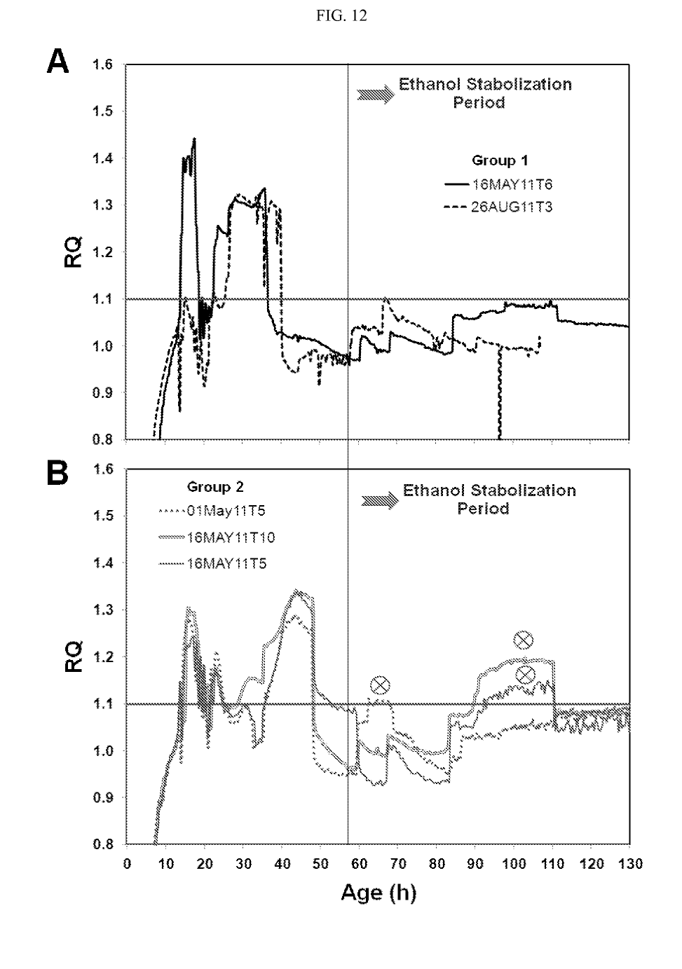

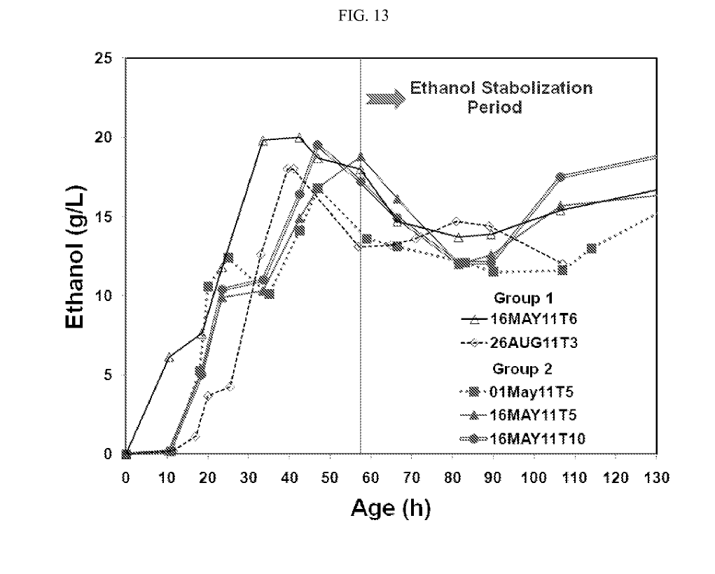

The RQ and ethanol profiles of five lots are shown in FIG. 12 and FIG. 13, respectively. Two different RQ control regimes are clearly recognized in FIG. 12. RQ values between 1.25 and 1.45 were applied in the period between 20 hours run time and the time reaching peak ethanol level. FIG. 13 showed that ethanol was built up and reached a peak of 17-22 g/L at the end of this period. RQ values between 0.95 and 1.15 were then applied afterwards. In order to observe the second RQ control regime, two lots (lots 16MAY11T6 and 26AUG11T3) were maintained at RQ values lower than 1.1 until the end of fermentation. These two lots are called Group 1. The other three lots (lots 01MAY11T5, 16MAY11T5, and 16MAY11T10) had at least a period (>3 hours) showing the RQ values greater than 1.1. Those three lots are called Group 2. FIG. 13 also showed that ethanol was maintained at 5-17 g/L during this period.

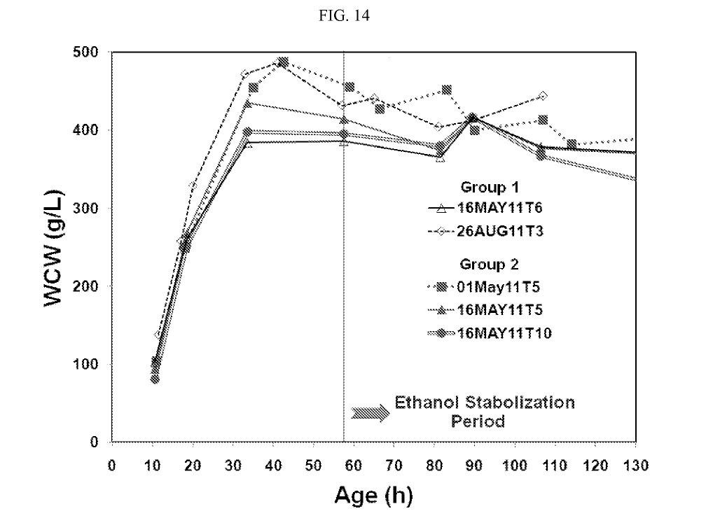

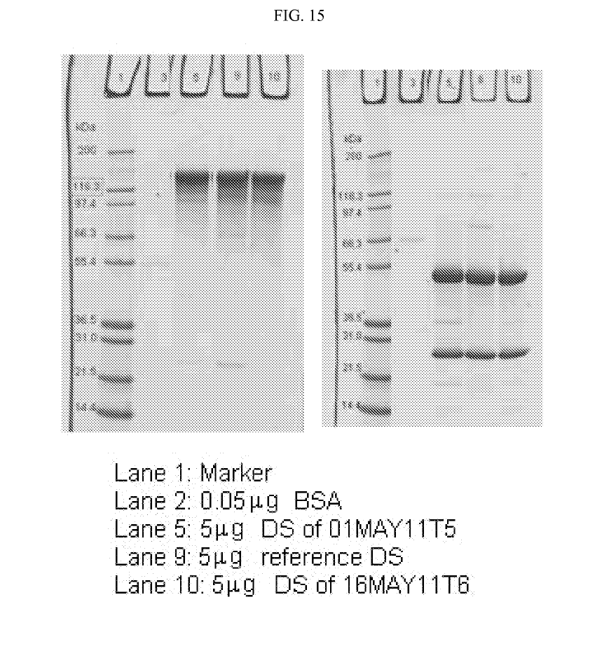

The WCW profiles are presented in FIG. 14, while titer and product quality results are presented below in Table 7. The WCW values reached peak values of 360-480 g/L at 30-40 hours run time when ethanol levels were approaching their peak. The WCW values were then maintained at 350-450 g/L afterwards. These profiles met the expectation as previously describe herein. Table 7 further demonstrated that the five lots produced comparable WB titer of 80-101 Normalized units at ˜132 hours. However, the 37/19 kD clipping variant did not reach a detectable level (<=1% mAb protein) in Group 1, but did show a detectable level in Group 2. Samples of Group 1 (Lot 16MAY11T6) and Group 2 (Lot 01MAY11T5) were run on a reducing SDS-PAGE gel and are presented for demonstration in FIG. 15.

TABLE 7
Summary of the RQ Testing Experiments

| | | | | |
|---|---|---|---|---|
| | Duration | WCW | WB Titer | |
| Lot # | (h) | (g/L) | (Normalized) | 37/19 kD Bands |

Group 1: RQ values <= 1.1 after 50 h

| | | | | |
|---|---|---|---|---|
| 16MAY11T6 | 132 | 371 | 100.7 | No detectable |
| 26AUG11T3 | 107 | 444 | 89.0 | No detectable |

Group 2: RQ values > 1.1 after 50 h

| | | | | |
|---|---|---|---|---|
| 01MAY11T5 | 131 | 389 | 85.5 | Presence |
| 16MAY11T5 | 132 | 369 | 88.3 | Presence |
| 16MAY11T10 | 132 | 334 | 79.9 | Presence |

In summary, Example 3 demonstrated two RQ control regimes of the fermentation process. The first RQ control regime at set point of 1.25-1.45 was applied to build up ethanol from 20 hours run time until reaching peak ethanol level of 18-22 g/L. The second RQ control regime at set point of 0.95-1.10 was applied to achieve relative steady ethanol and cell density afterwards. It should be observed that RQ values greater than 1.1 for a period greater than 3 hours would introduce a 37/19 kD clipping variant, which should be avoided during fermentation.

### Example 4

### Effect of RQ Control on Cell Growth and Protein Productivity of P. pastoris Fermentation: Case 2

Example 4 demonstrates the effects of respiratory quotient (RQ) control on product purity of the humanized anti-IL-6 antibody in Run 19JUN11. As mentioned in above Example 3, the desired product quality is less than detectable level (<1% of the antibody) of the 37/19 kD clipped variant. The media and process were previously described herein. The experiment was performed in six bioreactors.

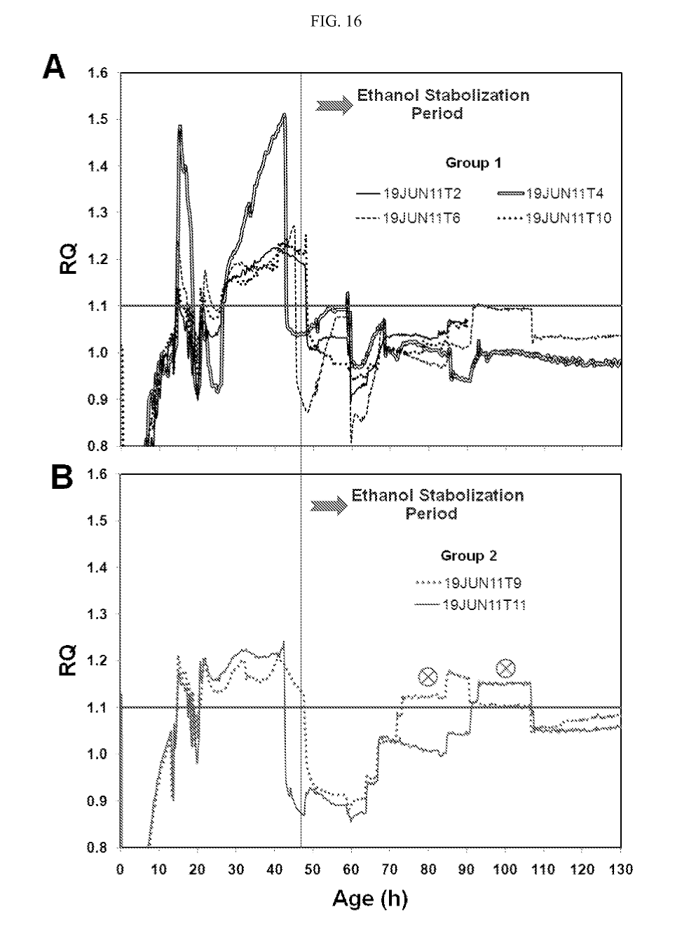

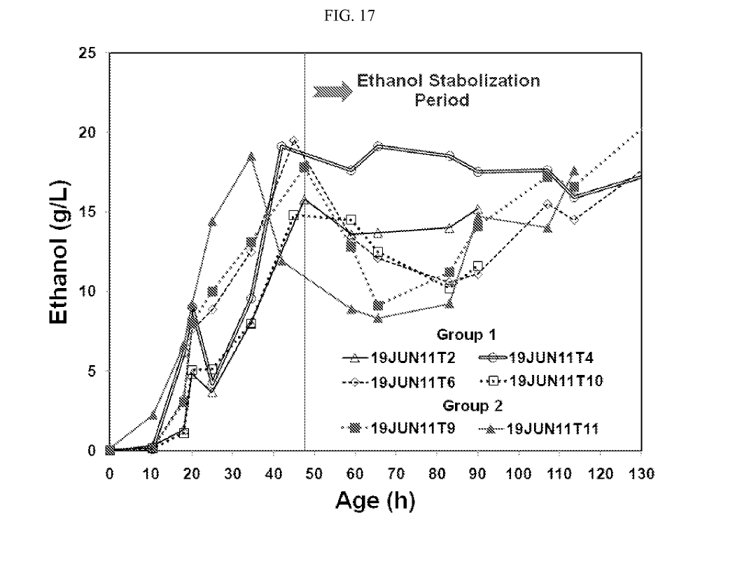

The RQ and ethanol profiles of six lots are presented in FIG. 16 and FIG. 17, respectively. Two different RQ control regimes can be clearly recognized in FIG. 16. RQ values between 1.20 and 1.50 were applied in the period between 25 hours run time and the time reaching peak ethanol level. FIG. 17 showed that ethanol was built up and reached a peak of 17-22 g/L at the end of this period. RQ values between 0.95 and 1.15 were then applied afterwards. Further observed the second RQ control regime, four lots (Lots 19JUN11T2, T4, T6 and T10) were maintained RQ values lower than 1.1 to the end of fermentation. These four lots are called Group 1. The other two lots (Lots 19JUN11T9 and T11) had at least a period (>3 hours) showing the RQ values greater than 1.1. Those three lots are called Group 2. FIG. 17 also showed that ethanol was maintained at 10-18 g/L during this period.

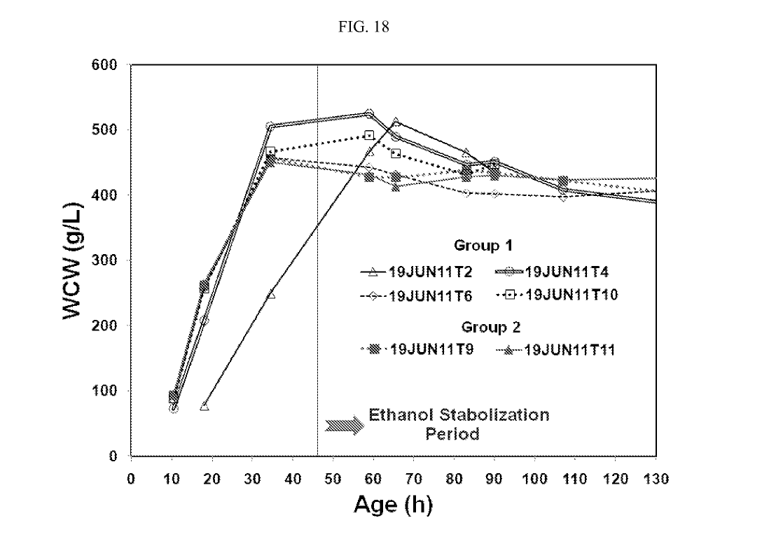

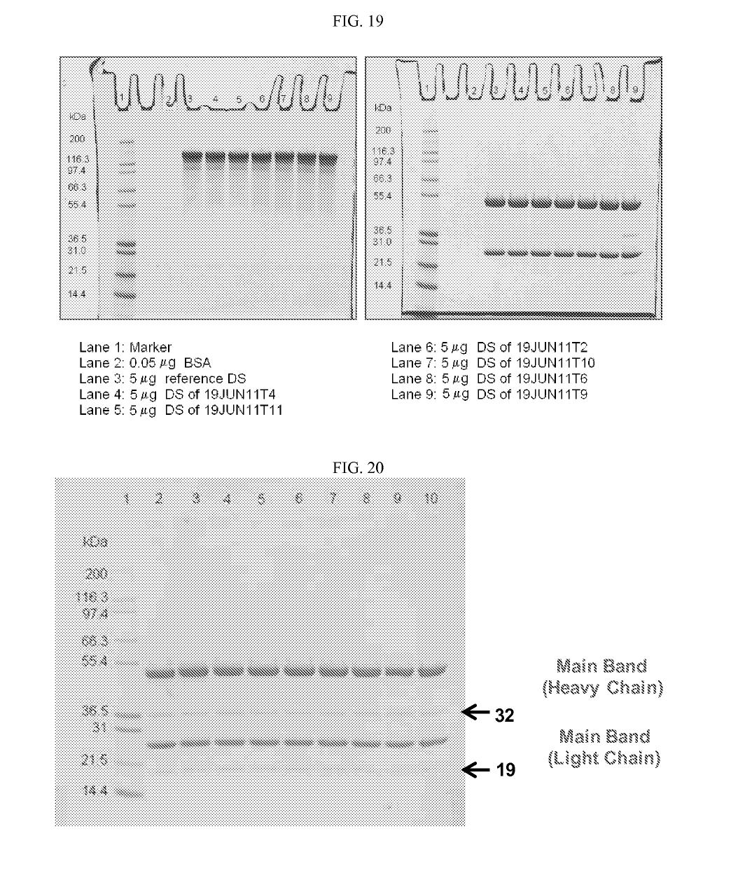

The WCW profiles are presented in FIG. 18, while titer and product quality results are presented below in Table 8. The WCW values reached peak values of 360-480 g/L at 30-40 hours run time when ethanol levels were approaching their peak. The WCW values were then maintained at 350-450 g/L afterwards. These profiles met the expectation as previously describe herein. Table 8 further demonstrated that six lots produced comparable WB titer of 71-98 normalized units at ˜131 hours. However, the 37/19 kD clipping variant did not reach detectable level (<=1% mAb protein) in Group 1, but was detected in Group 2. The SDS-PAGE gels are presented in FIG. 19.

TABLE 8
Summary of the RQ Testing Experiments

| | | | | |
|---|---|---|---|---|
| | Duration | WCW | WB Titer | |
| Lot # | (h) | (g/L) | (Normalized) | 37/19 kD Bands |

Group 1: RQ values <= 1.1 after 50 h

| | | | | |
|---|---|---|---|---|
| 19JUN11T2 | 90 | 440 | 72.8 | No detectable |
| 19JUN11T4 | 131 | 389 | 97.8 | No detectable |
| 19JUN11T6 | 131 | 407 | 89.0 | No detectable |
| 19JUN11T10 | 90 | 447 | 77.4 | No detectable |

Group 2: RQ values > 1.1 after 50 h

| | | | | |
|---|---|---|---|---|
| 19JUN11T9 | 131 | 406 | 71.3 | Presence |
| 19JUN11T11 | 131 | 426 | 86.1 | Presence |

In summary, Example 4 repeated the retrospective results of Example 3 in a side-by-side comparison experiment. It demonstrated that two RQ control regimes of the fermentation process. The first RQ control regime at set point of 1.2-1.5 was applied to build up ethanol from 20 hours run time until reaching peak ethanol level of 18-22 g/L. The second RQ control regime at set point of 0.95-1.10 was applied to achieve relative steady ethanol and cell density afterwards. It was observed that RQ values of greater than 1.1 for a period of greater than 3 hours introduced the 37/19 kD clipping variant.

### Example 5

### Identification of the Anti-IL-6 Antibody 37/19 kD Clipping Variant

To identify the 37/19 kD clipping variant observed in the fermentation lots reported in Examples 3 and 4, the antibody of 01MAY11T5 was used for protein N-terminal sequencing.

The samples were run on a reducing SDS-PAGE gel as shown in FIG. 20. After being transferred to a PROBLOTT® Mini membrane (Part number 401194, Applied Biosystems, Foster City, Calif.), the 37 kD and 19 kD bands were excised and extracted. The extracted samples were then N-terminal sequenced according to the manufacturer's protocol (LC 494 Procise Protein Sequencer, Applied Biosystems, Foster, Cali.). The light and heavy chains of the antibody were also N-terminal sequenced as the control.

The measured N-terminal amino acid sequences of light chain (LC) and heavy chain (HC) were as follows:

1. N-terminal of HC: E-V-Q-L-V-E-S-G-G-G (amino acid residues 1-10 of SEQ ID NO:12);

2. N-terminal of LC: A-I-Q-M-T-Q-S-P-S-S (amino acid residues 1-10 SEQ ID NO:3).

While N-terminal amino acid sequences of the extra bands of 37 kD and 19 kD showed the following results:

3. N-terminal of 37 kD band: E-V-Q-L-V-E-S-G-G-G (amino acid residues 1-10 of SEQ ID NO:12);

4. N-terminal of 19 kD band: T-Y-R-V-V-S-V-L-T-V (amino acid residues 302-311 of SEQ ID NO:12).

The above results demonstrate that the N-terminus of 37 kD band is identical to the heavy chain of the humanized anti-IL-6 antibody, while the N-terminal of 19 kD band is identical to the sequence starting from amino acid residue 302 (Thr) of the heavy chain as shown in SEQ ID NO:12. This indicates the two bands are the result of a clip between amino acid residue 301 (Ser) and amino acid residue 302 (Thr) of the heavy chain as shown in SEQ ID NO:12.

### Example 6

### Downstream Purification and Product Quality of Various P. pastoris Fermentation Conditions

Three 14 L lots (01MAY11T4, 01MAY11T5, and 16MAY11T6) were purified using a conventional 3-column downstream process consisting of Protein A capture and polishing steps. The three lots differ mainly in two conditions of the novel fermentation conditions, namely addition of hydroxyurea and respiratory quotient (RQ) control. Lot 16MAY11T6 is one of consistency runs of the novel fermentation process as described in Example 5, while RQ control was not applied to lots 01MAY11T5 and 01MAY11T4 yet, of which hydroxyurea was not added into lot 01MAY11T4 as shown in Example 2.

Results in below Table 9 suggest that the yield and purity of in-process pools of the three lots are in the range observed in a large number of similar lab runs. It could be concluded that the addition of hydroxyurea and RQ control strategy do not show significant impact on downstream column performance and product quality of in-process pools.

TABLE 9
Summary of downstream chromatography for Example 6

| | Capture | | Polishing 1 | | Polishing 2 | |
|---|---|---|---|---|---|---|
| | Yield, | Purity, | Yield, | Purity, | Yield, | Purity, |
| Lot # | % | % | % | % | % | % |
| 01MAY11T5 | 87 | 89.7 | 97 | 91.2 | 77 | 97.3 |
| 01MAY11T4 | 94 | 90.8 | 98 | 91.5 | 82 | 97.3 |
| 16MAY11T6 | 97 | 92.3 | 97 | 93.0 | 82 | 97.2 |

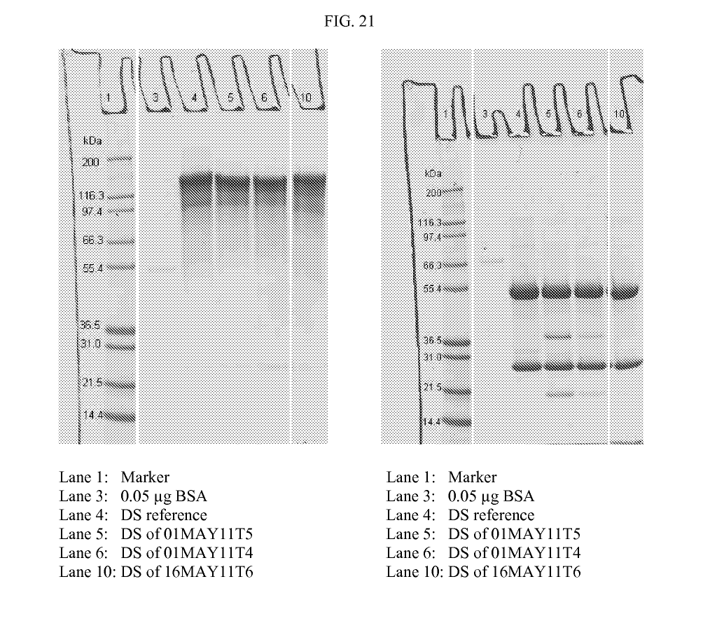

The SDS-PAGE gel and size-exclusion chromatography results of the antibody are presented in FIG. 21 and Table 10. The 37 kD and 19 kD bands were detected (>=1% antibody) in the antibody using the materials from the new fermentation process without RQ control (Lots 01MAY11T4 and 01MAY11T5). As shown in Example 5, these two bands are the result of a clip on heavy chain, thus are called 37/19 kD clip variant. Notably, the novel fermentation process with the new RQ control strategy (lot 16MAY11T6) showed that the 37/19 kD clipped variant was below the detectable level (<1% target antibody) or is “substantially free of cleavage” as determined by SDS-PAGE gel electrophoresis. Table 10 further demonstrated that the main peak of the antibody of all three lots was greater than 97.9% based on the size-exclusion chromatography, indicating the antibody can be purified from the fermentation broth using the conventional downstream process.

TABLE 10
Summary of size-exclusion chromatography
(SE-HPLC) results of the DS for Example 6

| | SE-HPLC | | |
|---|---|---|---|
| Lot # | Main | Pre-Main | Post-Main |
| 01MAY11T5 | 98.4 | 0.3 | 1.3 |
| 01MAY11T4 | 97.9 | 0.2 | 1.9 |
| 16MAY11T6 | 98.7 | 0.3 | 1.0 |
| DS Reference | 95.6 | 1.0 | 2.4 |

### Example 7

### Process Parameters of the Novel Fermentation Process for Antibody Production

Three consistent lots (16MAY11T6, 19JUN11T5, and 26AUG11T3) were performed to demonstrate the fermentation process for production of the humanized anti-IL-6 antibody. The media and processes utilized are as described herein.

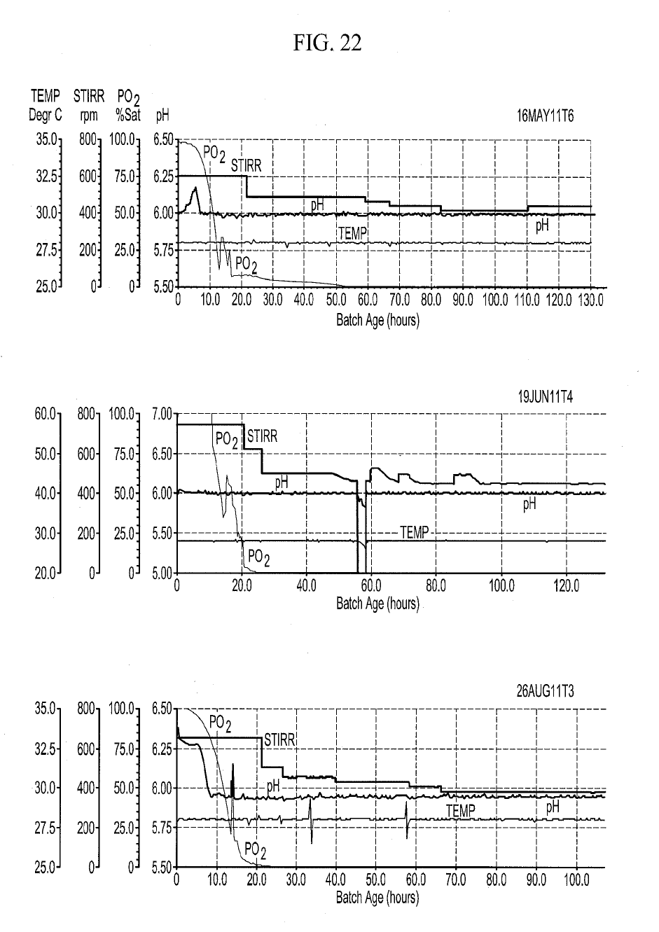

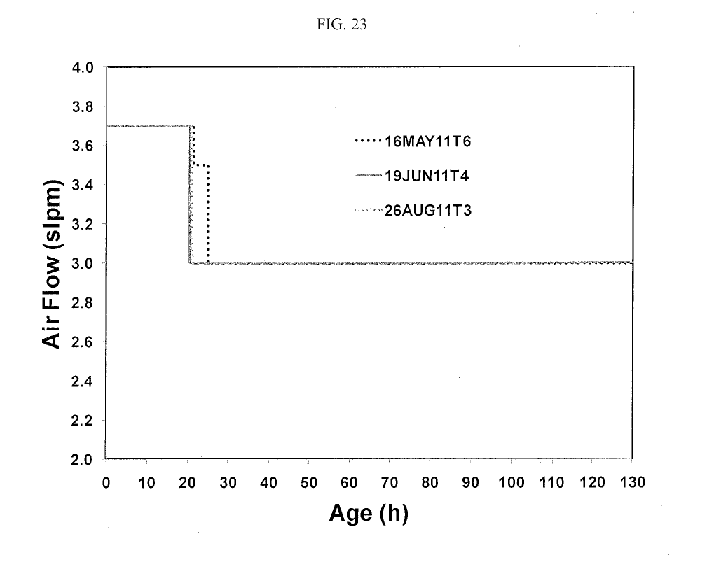

The engineering parameters including pH, temperature, agitation, airflow, and dissolved oxygen (measured by pO₂) are presented in FIG. 22 and FIG. 23. Overall, profiles of these engineering parameters met the parameter values as described herein.

1) Temperature and pH were maintained close to their set points (28° C. and pH 6.0) in the entire fermentation. It should be noted that oscillation of the pH was up to pH 6.3 in early fermentation (before 10 hours run time), which did not impact the fermentation performance.

2) A two step air flow setting was applied. Air flow was set at 3.7 SLPM (1 vvm) at fermentation start and shifted to 3.0 SLPM (0.8 vvm) two hours after the addition of hydroxyurea (˜20 hours run time) to enhance ethanol build up. In the development run (Lot 16MAY11T6), the second step airflow was originally designed as 3.5 SLPM and then adjusted to 3.0 SLPM on the demand of ethanol build up. The second step of airflow setting of the repeat runs (Lots 19JUN11T4 and 26AUG11T3) was fixed at 3.0 SLPM.

3) The bioreactor configuration of Lot 19JUN11T4 (three impellers with impeller to bioreactor diameter ratio of 0.33) is different from other two lots (16MAY11T6 and 26AUG11T3, two impellers with impeller to fermentor diameter ratio of 0.5). The initial agitations of these three lots were adjusted to have equivalent power to volume ratio.

4) The agitation was then adjusted to meet the RQ control regimes two hours after hydroxyurea addition (˜20 h run time). Reduced agitation speed from the initial setting was seen.

5) It should be noted that there was a two hour power outage at ˜55 hours run time in Lot 19JUN11T4, which did not impact fermentation performance.

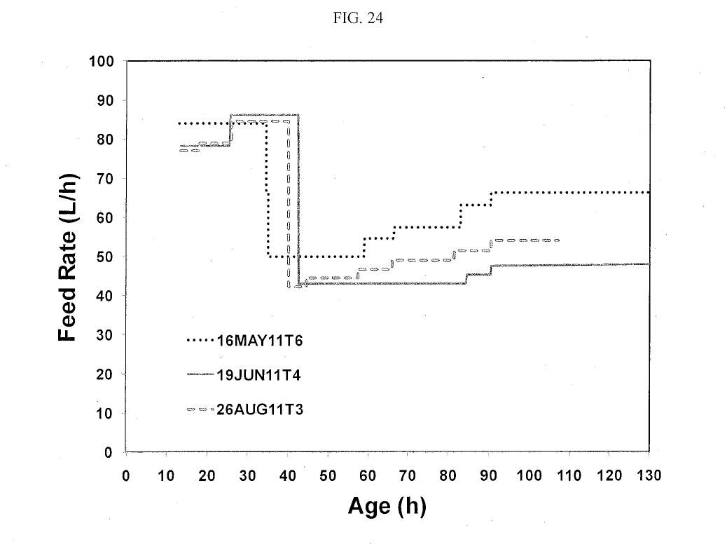

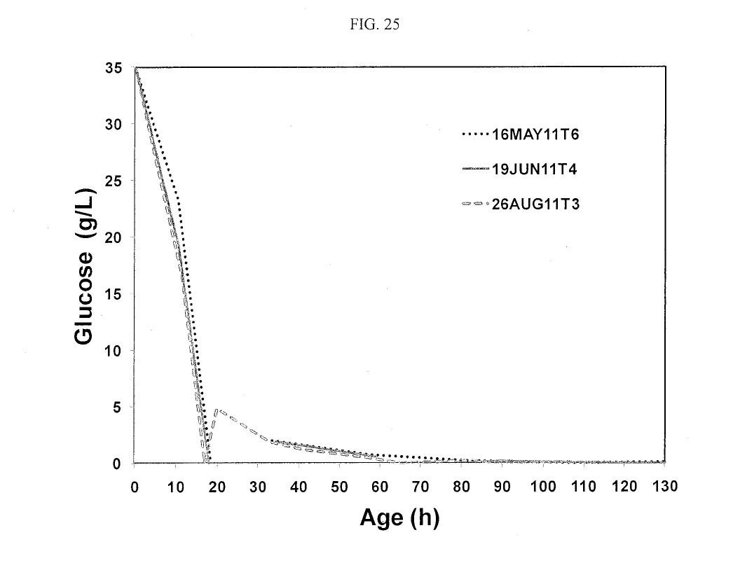

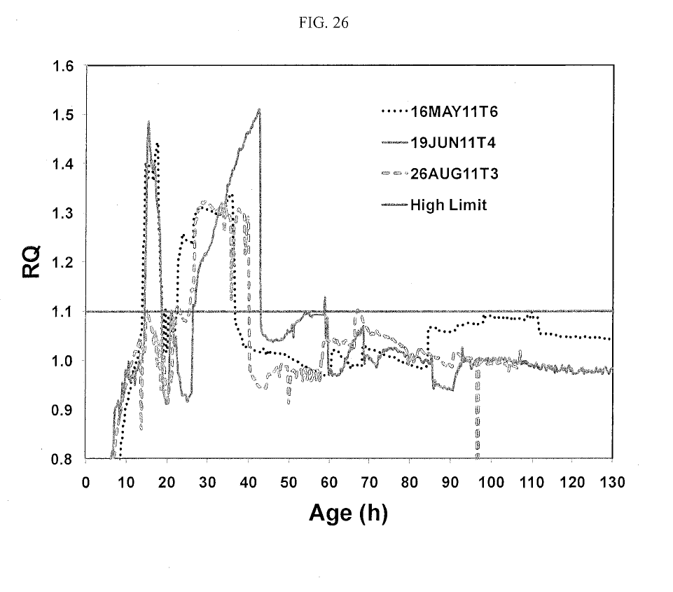

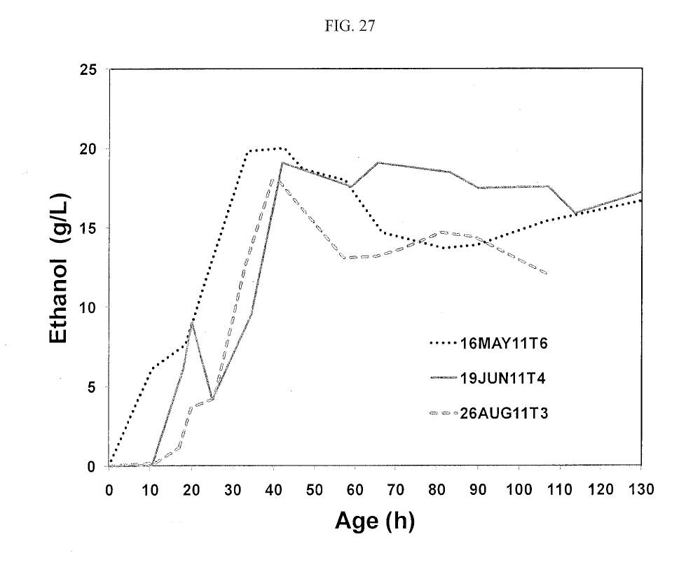

The control parameters including feeding rate, glucose level, RQ value and ethanol level are presented in FIG. 24, FIG. 25, FIG. 26 and FIG. 27. Overall, the profiles of these engineering parameters met the parameter values as described herein.

1) Feed rate was designed to keep a culture under glucose limit condition after feeding (glucose level close to zero). FIG. 24 showed that feeding was initiated at rate based on the glucose inlet flow of 11 g/L/h, reduced to 50% of initial rate when a culture reaching its peak ethanol level of 18-22 g/L, and increased by 5% of the current value approximately every other 12 h. FIG. 25 demonstrated glucose level reached zero before hydroxyurea addition (˜20 hours) and after 60 hours. It should be noted that the glucose values between 20 hours and 60 hours reflected the hydroxyurea interference for the glucose measurement by YSI (YSI Profiler).

2) RQ control was designed to keep the ethanol profile as described herein and as shown in FIG. 26 and FIG. 27. RQ values were initially monitored at 1.25 to 1.5 two hours after hydroxyurea addition (˜20 hours) until reaching peak ethanol level of 18-21 (at 35-45 hours run time). RQ values were then monitored at 0.95-1.1 that kept ethanol level at 10-17 g/L. In the later RQ control regime (RQ2), the high end of RQ control range can contribute to improved product quality. It was observed that a clip on heavy chain that caused the 37/19 kD bands could be generated when RQ >1.1 for a period (>3 hours). The low end of RQ control range can maintain ethanol level at certain level (10-17 g/L). Lower ethanol level usually correlated to high cell mass but low productivity.

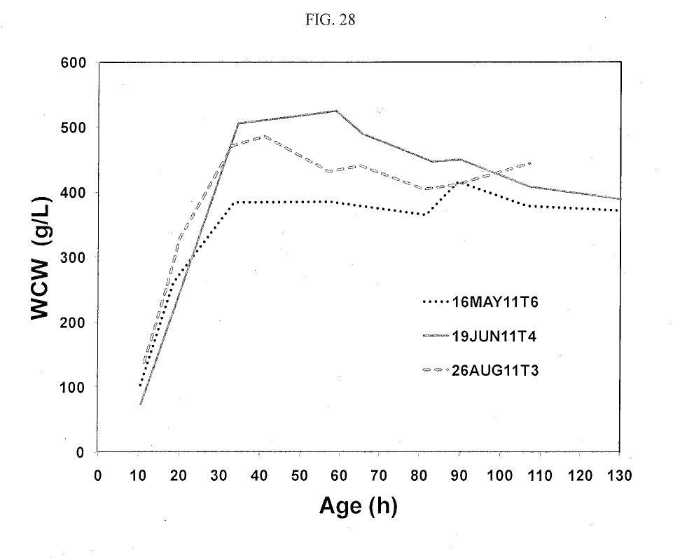

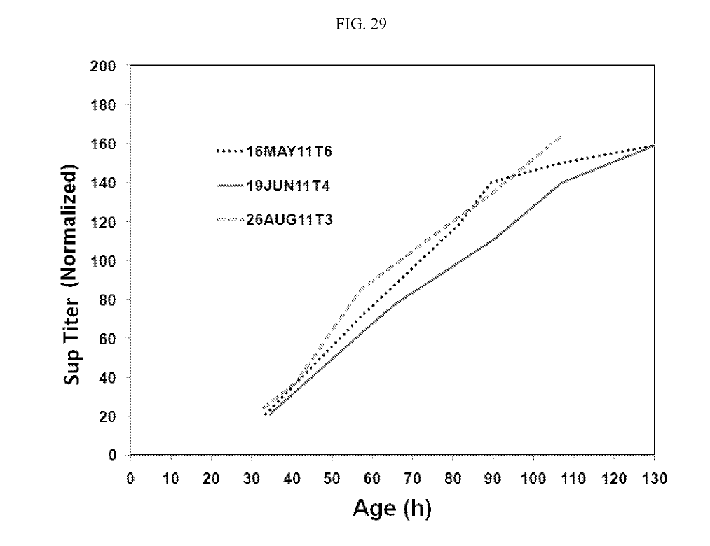

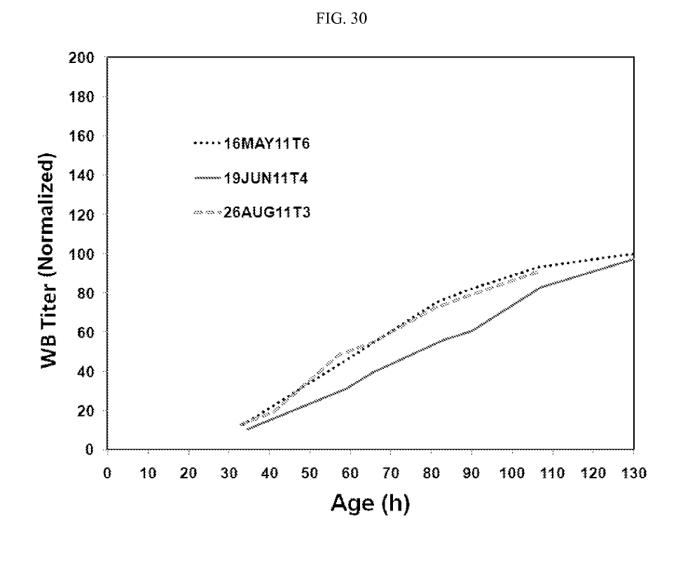

The performance parameters including wet cell weight (WCW), supernatant titer, and whole broth (WB) titer are presented in FIG. 28, FIG. 29 and FIG. 30. FIG. 28 demonstrated that WCW reached its peak of 380-550 g/L at 30-40 hours right before the cultures reaching the peak ethanol level of 18-22 g/L as previous shown in FIG. 27. Cultures were able to maintain WCW of 350-450 at the end of fermentation. FIG. 29 and FIG. 30 further demonstrated that supernatant and WB titer could be detected at ˜30 hours and continued to increase to the end of fermentation at 120-140 hours. At the harvest, Lot 26AUG11T3 produced WB titer of 91 normalized units at 107 hours and Lots 16MAY11T6 and 19JUN11T4 produced WB titer of 101 and 98 normalized units at 132 and 131 hours run time, respectively. The antibody of these three lots did not have a detectable 37/19 kD clipping variant.

In summary, three consistency lots (16MAY11T6, 19JUN11T4, and 26AUG11T3) demonstrated the fermentation process parameters as described herein. The new fermentation culture could produce WB titer of 90 normalized units at ˜110 hours and 100 normalized units at ˜130 hours run time without a detectable 37/19 kD clipping variant.

The complete disclosure of all patents, patent applications, and publications, and electronically available material (e.g., GENBANK® amino acid and nucleotide sequence submissions) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A method for producing an antibody or antigen-binding fragment thereof in Pichia pastoris comprising:
a) providing a population of cultured Pichia pastoris cells, wherein each cell comprises a DNA segment encoding a heavy chain polypeptide and a light chain polypeptide of the antibody or antigen-binding fragment operably linked to a promoter and a transcription terminator;
b) culturing the cells of step (a) under batch fermentation conditions;
c) culturing the cells of step (b) under fed-batch fermentation conditions comprising administering 2.0-5.0 g/L of hydroxyurea to the cell culture at about 12-30 hours of the fermentation process;
d) harvesting the cells of step (c) at about 100-140 hours of the fermentation process; and
e) recovering the antibody or antigen-binding fragment produced by the harvested cells of step (d).

2. The method of claim 1, wherein the promoter is a glyceraldehyde-3-phosphate (GAP) promoter.

3. The method according to claim 1, wherein the DNA segment encoding the heavy chain polypeptide and the light chain polypeptide are both operably linked to the same GAP promoter.

4. The method according to claim 1, wherein the DNA segment encoding the heavy chain polypeptide is operably linked to a first GAP promoter and the DNA segment encoding the light chain polypeptide is operably linked to a second GAP promoter.

5.-10. (canceled)

11. The method according to claim 1, wherein the antibody is a human, humanized or chimeric antibody.

12. (canceled)

13. (canceled)

14. The method according to claim 1, wherein about 2.0-4.5 g/L, about 2.0-4.0 g/L, about 3.0-4.0 g/L, about 2.5-5.0 g/L, about 2.1-2.9 g/L, about 2.2-2.8 g/L, about 2.6-2.8 g/L, about 2.5-2.8 g/L, about 2.6-2.9 g/L, about 2.3-2.7 g/L, about 2.4-2.6 g/L or about 2.5 g/L of hydroxyurea is added at about 12-30 hours or about 16-22 hours of the fermentation process.

15. The method according to claim 1, wherein the hydroxyurea is added at about 14-19 hours or at about 16-21 hours of the fermentation process.

16. The method according to claim 1, further comprising the step of adjusting a first respiratory quotient (RQ1) to about 1.36-1.6, to about 1.36-1.45, to about 1.45-1.6, or to about 1.4-1.5 at about 16/21-32/48 hours of the fermentation process.

17. The method according to claim 1, further comprising the step of increasing the concentration of ethanol to about 18-22 g/L or about 19-21 g/L of the cell culture at about 16/21-32/48 hours of the fermentation process.

18. (canceled)

19. The method according to claim 1, further comprising the step of adjusting a second respiratory quotient (RQ2) to about 0.8-1.06, to about 0.85-1.06, to about 0.90-1.06, to about 0.95-1.06 or less than 1.07 at about 32/48-100/140 hours of the fermentation process.

20. The method according to claim 1, further comprising the step of stabilizing the ethanol concentration of the cell culture to a concentration of about 5-17 g/L, to about 8-17 g/L, about 9-17 g/L, about 10-17 g/L, about 11-17 g/L, about 12-17 g/L about 8-16 g/L, about 8-15 g/L, about 8-14 g/L or about 8-13 g/L at about 32/48-100/140 hours of the fermentation process.

21. The method according to claim 16, further comprising the step of increasing the concentration of ethanol to about 18-22 g/L or about 19-21 g/L of the cell culture at about 16/21-32/48 hours of the fermentation process.

22. The method according to claim 16, further comprising the step of adjusting a second respiratory quotient (RQ2) to about 0.8-1.06, to about 0.85-1.06, to about 0.90-1.06, to about 0.95-1.06 or less than 1.07 at about 32/48-100/140 hours of the fermentation process.

23. The method according to claim 17, further comprising the step of adjusting a second respiratory quotient (RQ2) to about 0.8-1.06, to about 0.85-1.06, to about 0.90-1.06, to about 0.95-1.06 or less than 1.07 at about 32/48-100/140 hours of the fermentation process.

24. The method according to claim 17, further comprising the step of stabilizing the ethanol concentration of the cell culture to a concentration of about 5-17 g/L, to about 8-17 g/L, about 9-17 g/L, about 10-17 g/L, about 11-17 g/L, about 12-17 g/L about 8-16 g/L, about 8-15 g/L, about 8-14 g/L or about 8-13 g/L at about 32/48-100/140 hours of the fermentation process.

25. The method according to claim 19, further comprising the step of stabilizing the ethanol concentration of the cell culture to a concentration of about 5-17 g/L, to about 8-17 g/L, about 9-17 g/L, about 10-17 g/L, about 11-17 g/L, about 12-17 g/L about 8-16 g/L, about 8-15 g/L, about 8-14 g/L or about 8-13 g/L at about 32/48-100/140 hours of the fermentation process.

26. The method according to claim 17, further comprising the step of adjusting a first respiratory quotient (RQ1) to about 1.36-1.6, to about 1.36-1.45, to about 1.45-1.6, or to about 1.4-1.5 at about 16/21-32/48 hours of the fermentation process.

27. The method according to claim 19, further comprising the step of adjusting a first respiratory quotient (RQ1) to about 1.36-1.6, to about 1.36-1.45, to about 1.45-1.6, or to about 1.4-1.5 at about 16/21-32/48 hours of the fermentation process.

28. The method according to claim 23, further comprising the step of adjusting a first respiratory quotient (RQ1) to about 1.36-1.6, to about 1.36-1.45, to about 1.45-1.6, or to about 1.4-1.5 at about 16/21-32/48 hours of the fermentation process.

29. The method according to claim 25, further comprising the step of adjusting a first respiratory quotient (RQ1) to about 1.36-1.6, to about 1.36-1.45, to about 1.45-1.6, or to about 1.4-1.5 at about 16/21-32/48 hours of the fermentation process.

30. The method according to claim 20, further comprising the step of adjusting a first respiratory quotient (RQ1) to about 1.36-1.6, to about 1.36-1.45, to about 1.45-1.6, or to about 1.4-1.5 at about 16/21-32/48 hours of the fermentation process.

31. The method according to claim 30, further comprising the step of adjusting a second respiratory quotient (RQ2) to about 0.8-1.06, to about 0.85-1.06, to about 0.90-1.06, to about 0.95-1.06 or less than 1.07 at about 32/48-100/140 hours of the fermentation process.

32. The method according to claim 28, further comprising the step of stabilizing the ethanol concentration of the cell culture to a concentration of about 5-17 g/L, to about 8-17 g/L, about 9-17 g/L, about 10-17 g/L, about 11-17 g/L, about 12-17 g/L about 8-16 g/L, about 8-15 g/L, about 8-14 g/L or about 8-13 g/L at about 32/48-100/140 hours of the fermentation process.
